(19) United States
(12) Patent Application Publication
(10) Pub. No.: US 2020/0138711 A1
(43) Pub. Date: May 7, 2020

(54) INJECTABLE HYDROGELS AND USES THEREOF

(71) Applicants: UNIVERSITE DE STRASBOURG, STRASBOURG (FR); CENTRE NATIONAL DE LA RECHERCHE SCIENTIFIQUE, PARIS (FR); IHU STRASBOURG - INSTITUT HOSPITALO-UNIVERSITAIRE DE STRASBOURG, STRASBOURG (FR)

(72) Inventors: Luisa DE COLA, STRASBOURG (FR); Giuseppe ALONCI, STRASBOURG (FR); Silvana PERRETTA, STRASBOURG (FR); Pietro RIVA, CESANO MADERNO (IT); Federica FIORINI, PADOVA (IT)

(21) Appl. No.: 16/478,500

(22) PCT Filed: Jan. 17, 2018
(86) PCT No.: PCT/EP2018/051130

§ 371 Date: Jul. 17, 2019

Related U.S. Application Data
(60) Provisional application No. 62/447,056, filed on Jan. 17, 2017.

(30) Foreign Application Priority Data
Jan. 12, 2017 (EP) 17306692.9
Sep. 15, 2017 (EP) 17306195.3

(51) Int. Cl. A61K 9/06; C08G 73/02; A61K 9/51; A61K 9/00

(52) U.S. Cl. CPC A61K 9/06; A61K 9/0019; A61K 9/5123; C08G 73/028

(57) ABSTRACT

The invention relates to a hydrogel, in particular degradable or non degradable, comprising monomers of formula (I) and organosilica particles or porous silicon particles covalently bound thereto, optionally with non covalently bound organosilica and/or silicon particles mixed therewith, in particular degradable organosilica nanoparticles or core-shell nanocapsules; pharmaceutical, veterinary or cosmetic compositions thereof; and uses thereof as a medicament. The present invention finds applications in the therapeutic and diagnostic medical technical fields and also in cosmetic and veterinary technical fields.

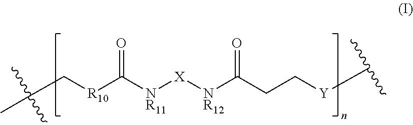

### PRIORITY

This application relates to U.S. Provisional patent Application No. 62/447,056 filed on 17 Jan. 2017; European Provisional Patent Application no EP 17306195.3 filed on 15 Sep. 2017; European Provisional Patent Application no EP 17306692.9 filed on 1 Dec. 2017; the entire contents of each of which are hereby incorporated by reference.

### FIELD

The invention relates to a hydrogel, in particular degradable or non degradable, comprising monomers of formula (I) and organosilica particles or porous silicon particles covalently bound thereto, optionally with non covalently bound organosilica particles and/or silicon particles mixed therewith, in particular degradable organosilica nanoparticles or core-shell nanocapsules; pharmaceutical, veterinary or cosmetic compositions thereof; and uses thereof as a medicament.

The present invention finds applications in the therapeutic and diagnostic medical technical fields and also in cosmetic and veterinary technical fields.

### BACKGROUND

Biocompatible soft materials, and in particular hydrogels and liquids that can form interlayers between tissues, have been recently used in surgery to facilitate resection of tumors.

They find applications as submucosal fluid cushions (SFC), to avoid perforation and thus to facilitate endoscopic submucosal dissection (ESD). ESD is a clinical procedure applied for early stage neoplastic lesions in the gastrointestinal tract that allows en bloc resection of large lesions. During surgery it is necessary to lift the mucosa and to prevent the occurrence of damages to deeper tissues.

The injection of materials, which can gel in situ forming a protective layer between the part to be removed and the healthy tissue, has been proposed as a way to avoid perforation and thus to facilitate ESD.

Even though several injection solutions have been proposed and tested, normal saline solution (NS) is the most commonly used in the clinic because of its low cost and ease of use. However, it is hampered by low mucosal elevations, making the procedure difficult and often resulting in electrocautery damage of the muscularis (i.e. the thin layer of muscle of the GI tract, which is located underneath the submucosa). In addition, its rapid absorption in the surrounding tissues requires repeated injections for extensive resection.^{[8]}

Various substances, including glycerol,^{[10]} hydroxypropyl methylcellulose^{[11]} and hyaluronic acid^{[12]} have been exploited to achieve sustained mucosal elevation and avoid injuries to the muscularis propria. Although hyaluronic acid solution is one of the best options,^{[12]} it has been shown to induce a serious side effect which corresponds to a stimulation of the growth of residual tumors proliferation in animal models.^{[13]} Moreover, a large amount of hyaluronic acid is necessary to create a SFC and its use is associated with high costs (US $550.58/g) and a general lack of availability.^{[7]}

In the last years, injectable hydrogels have brought a shift in the search for the optimal SFC material towards the development of solutions that rely on in situ gel formation. For example, a photo-crosslinked chitosan hydrogel has been recently reported as a submucosal injection agent: mucosal elevation was created after the injection of the chitosan viscous solution, which was crosslinked in situ via UV irradiation, resulting in an insoluble hydrogel.

However, the use of UV light for the photoinitiated radical polymerization may be difficult in hard-to-reach areas and resulted somehow inconvenient as performed by the authors: was irradiated with UV light for a total of 5 min (30 s each at 10 different places by using an UV light-fiber through the endoscopic accessory channel and UV lamp system). Moreover, the authors mentioned that UV irradiation may be associated with inflammation of the residual tissue.

Thermoresponsive polymers, or thermogels, have been investigated as well for ESD applications, such as the recently proposed water solution of a PEG/PLGA-based temperature-sensitive polymer. However, many of these materials have been shown to clog inside long delivery tools at normal body temperature.

There is therefore a real need to find a compound which allows more efficient treatment and/or effective treatment and/or a compound which is not rapidly absorbed after injection and/or a compound that would reduce the number of injections.

There is also a real need to find a product/compound that could be clinically applicable, for example be biocompatible, easily injectable, able to provide a prolonged and thick SFC to allow the ESD procedure safely, cost-effective.

Biocompatible soft materials, and in particular hydrogels have been also proposed as dressing for example for topical wound. For example, hydrogels are particularly useful on superficial and deep chronic wounds, ulcers, leg ulcers, restorative and reconstructive surgery, sluggish wounds, dermabrasion, severe sunburn, superficial and deep burns of the second degree. Such dressings are commercially available, for example, Askina Gel sold by B Braun, Duoderm Hydrogel sold by Convatec, Hydrosorb sold by Hartmann, IntraSite Gel marketed by Smith & Nephew, Normgel sold by Mölnlycke, Purilon sold by Coloplas and Urgo hydrogel sold by Urgo.

However, known hydrogels have limited spectra of uses and are particularly designed to fit to specific wound and/or to be used in particular environments. In addition, known hydrogels are most of the time roughly applied onto the surface of the wound and cannot be injected at the wound and/or lesion site.

In addition, when the lesion and/or the wound is located between tissues and/or at the interface of tissues, for example in the gastrointestinal tract and/or at chirurgical site within the body of a mammal, the known hydrogels can, most of the time, not be used due to their rheological and/or biocompatible properties. In particular, most of the known hydrogels used as wound dressing are not biocompatible and/or biodegradable in-situ.

In addition, the known hydrogels are reticulated previously to their use and thus cannot be injected, for example with a needle, due to their viscosity.

There is thus a real need to find a product/compound that could be used as fillers and/or materials that could be injected/applied onto/into a wound. There is also a need to find product/compound, that could be an adaptive to fit to any wound whatever its form and/or size and/or localization.

There is also a need to find a biocompatible and/or biodegradable product that could avoid/reduce after injection side effect such as inflammation and be naturally resorbed and/or degraded after injection.

There is also a real need to find a product/compound that could be controlled and be tunably biodegradable, and/or could possibility release active components and/or molecules, for example to prevent bacterial infection and/or enhance healing at the lesion site.

### Definitions

To facilitate an understanding of the present invention, a number of terms and phrases are defined below:

As used herein other than the claims, the terms “a,” “an,” “the,” and/or “said” means one or more. As used herein in the claim(s), when used in conjunction with the words “comprise,” “comprises” and/or “comprising,” the words “a,” “an,” “the,” and/or “said” may mean one or more than one. As used herein and in the claims, the terms “having,” “has,” “is,” “have,” “including,” “includes,” and/or “include” has the same meaning as “comprising,” “comprises,” and “comprise.” As used herein and in the claims “another” may mean at least a second or more. As used herein and in the claims, “about” refers to any inherent measurement error or a rounding of digits for a value (e.g., a measured value, calculated value such as a ratio), and thus the term “about” may be used with any value and/or range.

The phrase “a combination thereof” “a mixture thereof” and such like following a listing, the use of “and/or” as part of a listing, a listing in a table, the use of “etc” as part of a listing, the phrase “such as,” and/or a listing within brackets with “e.g.,” or i.e., refers to any combination (e.g., any sub-set) of a set of listed components, and combinations and/or mixtures of related species and/or embodiments described herein though not directly placed in such a listing are also contemplated. Such related and/or like genera(s), sub-genera(s), specie(s), and/or embodiment(s) described herein are contemplated both in the form of an individual component that may be claimed, as well as a mixture and/or a combination that may be described in the claims as “at least one selected from,” “a mixture thereof” and/or “a combination thereof.”

In general, the term “substituted” whether preceded by the term “optionally” or not, and substituents contained in formulae of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds.

As used herein, the term “alkyl”, refers to straight and branched alkyl groups. An analogous convention applies to other generic terms such as “alkenyl”, “alkynyl” and the like. In certain embodiments, as used herein, “lower alkyl” is used to indicate those alkyl groups (substituted, unsubstituted, branched or unbranched) having about 1-6 carbon atoms. Illustrative alkyl groups include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “C₁₋ₓ alkylenyl”, as used herein, refers to a linear or branched saturated divalent radical consisting solely of carbon and hydrogen atoms, having from one to x carbon atoms, having a free valence “-” at both ends of the radical. Likewise, the term “C₁₋ₓ heteroalkylenyl”, as used herein, refers to a linear or branched saturated divalent C₁₋ₓ alkylenyl radical as defined above, comprising at least one heteroatom selected from O, N, or S, and having a free valence “-” at both ends of the radical. When the C₁₋ₓalkylenyl or C₁₋ₓ heteroalkylenyl is optionally substituted, at least one of the H atoms may be replaced by a substituent such as halogen or —OR where R may represent C1-6alkyl.

The term “ethenylenyl”, as used herein, refers to the divalent radical —CH═CH—. When the ethenylenyl is optionally substituted, one or both the H atoms may be replaced by a substituent such as halogen or —OR where R may represent C1-6alkyl.

In general, the term “aromatic moiety” or “aryl”, as used herein, refers to stable substituted or unsubstituted unsaturated mono- or polycyclic hydrocarbon moieties having preferably 3-14 carbon atoms, comprising at least one ring satisfying the Hackle rule for aromaticity. Examples of aromatic moieties include, but are not limited to, phenyl, indanyl, indenyl, naphthyl, phenanthryl and anthracyl.

The term “halogen” as used herein refers to an atom selected from fluorine, chlorine, bromine and iodine.

As used herein, the term “independently” refers to the fact that the substituents, atoms or moieties to which these terms refer, are selected from the list of variables independently from each other (i.e., they may be identical or the same).

As used herein, the term “template” or “supramolecular template” refers to a self-aggregation of ionic or non-ionic molecules or polymers that have a structure directing function for another molecule or polymer.

As used herein, the term “and/or” means any one of the items, any combination of the items, or all of the items with which this term is associated.

As used herein, the term “about” can refer to a variation of ±5%, ±10%, ±20%, or ±25%, of the value specified. For example, “about 50” percent can in some embodiments carry a variation from 45 to 55 percent. For integer ranges, the term “about” can include one or two integers greater than and/or less than a recited integer. Unless indicated otherwise herein, the term “about” is intended to include values, e.g., weight percents, proximate to the recited range that are equivalent in terms of the functionality of the individual ingredient, the composition, or the embodiment.

As will be understood by the person of ordinary skill in the art, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those persons of ordinary skill in the art utilizing the teachings of the descriptions herein. It is also understood that such values inherently contain variability necessarily resulting from the standard deviations found in their respective testing measurements.

As will be understood by a person of ordinary skill in the art, for any and all purposes, particularly in terms of providing a written description, all ranges recited herein also encompass any and all possible subranges and combinations of subranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by a person of ordinary skill in the art, all language such as “up to,” “at least,” “greater than,” “less than,” “more than,” “or more,” and the like, include the number recited and such terms refer to ranges that can be subsequently broken down into subranges as discussed above. In the same manner, all ratios recited herein also include all subratios falling within the broader ratio. Accordingly, specific values recited for radicals, substituents, and ranges, are for illustration only; they do not exclude other defined values or other values within defined ranges for radicals and substituents.

A person of ordinary skill in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

An “effective amount” refers to an amount effective to treat a disease, disorder, and/or condition, or to bring about a recited effect. For example, an amount effective can be an amount effective to reduce the progression or severity of the condition or symptoms being treated. Determination of a therapeutically effective amount is well within the capacity of persons of ordinary skill in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes medical, therapeutic, and/or prophylactic administration, as appropriate.

The term “responsively disintegratable”, when referring to the shell of the nanocapsule system according to the invention, refers to the property of a material or particle that undergoes degradation (i.e., breakdown of the structural integrity of the material or particle) triggered by a particular signal. The signal can be, for example, a change in pH (either an increase or decrease), a change in redox potential, the presence of reduction or oxidation agent, the presence of UV, visible or near infrared light, ultrasounds, electromagnetic radiation, an enzymatic cleavage, a change in temperature, etc.

The term “responsively cleavable”, when referring to a chemical bond, polymer fragment or linking group, refers to a covalent bond, polymer fragment or linking group that is cleaved upon application of one of the aforementioned particular signals. Generally speaking, the presence of a responsively cleavable bond, polymer fragment or linker moiety within a siliconoxide nanocapsule shell of the invention, confers to the nanocapsule shell its disintegratable properties (the property of structurally breaking down upon application of a specific signal/stimulus, akin to “self-destructive” behavior). Conversely, the term “stable covalent bond” refers to a covalent bond that is not cleaved in the environment to which it is exposed and/or upon application of one of the aforementioned particular signals. In that sense, the term “stable covalent bond” may be used interchangeably with “non-responsively cleavable covalent bond”.

As used herein, the term “hydrogel” refers to polymers comprising a solid polymer lattice and an interstitial aqueous phase.

As used herein, the term “degradable hydrogel” refers to hydrogels comprising at least one crosslinker within its structure, which can be cleaved upon application of a suitable signal/stimulus, or by biodegradation of the linker, resulting in the breakdown of the hydrogel crosslinked structure. For example, the hydrogel may comprise a redox-responsive crosslinker, such as cystamine crosslinker, which can be cleaved in response to a change in the redox potential of the environment. For example, a cystamine crosslinker may cleave in response to a variation in glutathione concentration in the surrounding environment. In yet another example, the hydrogel may comprise a pH-responsive crosslinker, such as an imine-bond containing crosslinker, which can be cleaved in response to a change in pH of the environment. The hydrogel may be said to be biodegradable when the environment is a physiological environment, and/or when the hydrogel contains at least one crosslinker which can undergo cleavage by biological means (bacteria, enzymes, etc.). Examples of degradable linkers being sugars, hyaluronic acid derivatives, aminoacids and peptides.

As used herein, the term “biological polymer” or “biopolymer” refers to polymers produced by living organisms, or synthetic mimics of those. There are three main classes of biopolymers, classified according to the monomeric units used and the structure of the biopolymer formed: polynucleotides (RNA and DNA), which are long polymers composed of 4 or more, for example 13 or more nucleotide monomers; polypeptides, which are short polymers of amino acids; and polysaccharides, which are often linear bonded polymeric carbohydrate structures.

As used herein, the term “biodegradable polymer” refers to natural or synthetic polymers, which can undergo chemical dissolution by biological means (bacteria, enzymes, etc.)

As used herein, the term “surfactant” refers to an ordered supramolecular assembly of surfactant or block copolymer molecule micelles, with translation symmetry between about 2 and about 50 nm.

As used herein, the term “cleavable” refers both to the reversible/biodegradable nature of linkers such as *—R¹-L₁-R²—* and #—R³-L₂-R⁴-#, as defined herein, triggering the decomposition/disintegration of the hydrogel framework material and/or degradable organosilica material (nanoparticles/nanocapsules) that may be bound to the hydrogel polymer network. As such, the linker may contain a dynamic covalent bond.

As used herein, the term “dynamic covalent bond” refers to any covalent chemical bond possessing the capacity to be formed and broken under equilibrium control. In this sense, they can be intended as “reversible” covalent bonds. [29]

As used herein, a “bioactive macromolecule” refers to a macromolecular biomolecule in an undenatured state, which still shows a conformation suited to carry on its supposed biological activity.

As used herein, a “biomolecule” refers to a naturally-occurring molecule (e.g., a compound) comprising of one or more chemical moiety(s) [“specie(s),” “group(s),” “functionality(s),” “functional group(s)” ], including but not limited to, polynucleotides (RNA and DNA), which are long polymers composed of 4 or more, for example 13 or more nucleotide monomers; polypeptides, which are short polymers of amino acids; proteins; and polysaccharides, which are often linear bonded polymeric carbohydrate structures, or a combination thereof. Examples of a macromolecule includes, an enzyme, an antibody, a receptor, a transport protein, structural protein, a prion, an antibiological proteinaceous molecule (e.g., an antimicrobial proteinaceous molecule, an antifungal proteinaceous molecule), or a combination thereof.

As used herein a “proteinaceous molecule,” proteinaceous composition,” and/or “peptidic agent” comprises a polymer formed from an amino acid, such as a peptide (i.e., about 3 to about 100 amino acids), a polypeptide (i.e., about 101 or more amino acids, such as about 50,000 or more amino acids), and/or a protein. As used herein a “protein” comprises a proteinaceous molecule comprising a contiguous molecular sequence of three amino acids or greater in length, matching the length of a biologically produced proteinaceous molecule encoded by the genome of an organism. Examples of a proteinaceous molecule include an enzyme, an antibody, a receptor, a transport protein, a structural protein, or a combination thereof. Examples of a peptide (e.g., an inhibitory peptide, an antifungal peptide) of about 3 to about 100 amino acids (e.g., about 3 to about 15 amino acids). A peptidic agent and/or proteinaceous molecule may comprise a mixture of such peptide(s) (e.g., an aliquot of a peptide library), polypeptide(s) and/or protein(s), and may also include materials such as any associated stabilizer(s), carrier(s), and/or inactive peptide(s), polypeptide(s), and/or protein(s).

### DESCRIPTION OF EXEMPLARY EMBODIMENTS

As discussed above, it may be advantageous to provide an injectable product that is biocompatible and biodegradable, and compound that could be used as to fit any wound whatever its form and/or size and/or localization. In this context, there is provided herein efficient polymerization methods able to address or accomplish this goal. In one aspect, there is provided a hydrogel comprising monomers of formula (I):

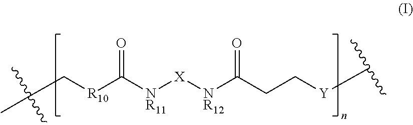

wherein

n is an integer representing the number of monomers (I) in the hydrogel polymer;

for each occurrence of the bracketed structure n, Y independently represents:
a molecular crosslinker for connecting at least a monomer of formula (I) in the framework to at least another monomer of formula (I) in another framework through a linker having the following structure:

*—R¹-L₁-R²—*;
wherein:
each occurrence of *—R¹-L₁-R²—* independently represents a responsively cleavable moiety or a non-cleavable moiety;
each occurrence of * denotes a point of attachment of the linker to a monomer of formula (I) in the hydrogel's framework;
each occurrence of L₁ independently represents a responsively cleavable covalent bond, a moiety containing a responsively cleavable covalent bond and/or a stable covalent bond;
R¹ and R², for each occurrence, independently represent an optionally substituted C1-20 alkylenyl moiety, an optionally substituted C1-20heteroalkylenyl moiety, an optionally substituted ethenylenyl moiety, —C≡C— or an optionally substituted phenyl moiety, wherein the C1-20 alkylenyl, C1-20 heteroalkylenyl or ethenylenyl moiety may bear one or more substituents selected from halogen or —OR where R may represent H or C1-6 alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
wherein *—R¹-L₁-R²—* may independently comprise sugar derivatives such as glucose, lactose or mannose derivatives, hyaluronic acid derivatives, collagene, aminoacids or peptides;
or
O, S or a group of formula

*—R₇(R₈)—*
wherein
the * symbols denote the points of attachment of Y within the monomer backbone of formula (I);
R⁷ represents N,
R⁸ represents an optionally substituted C1-20 alkyl, C1-20alkenyl or C1-20alkynyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety, wherein each of the foregoing C1-20 alkyl, C1-20alkenyl, C1-20alkynyl, C1-20heteroalkyl or C1-20alkylphenyl moieties may bear one or more substituents selected from halogen, —OR, —CO₂R or —N(Rp)2; where R may represent H or C1-6alkyl and each occurrence of Rp may independently represent H or C1-6alkyl; and the phenyl moiety may bear one or more substituents independently selected from halogen, C1-6alkyl, —NO₂, —CN, isocyano, —ORp, —N(Rp)2 wherein each occurrence of Rp independently represents H, C1-6alkyl or C1-6 alkoxy; wherein R⁸ may be optionally crosslinked to another monomer of formula (I) in another hydrogel polymer chain; or
a hyaluronic acid, alginic acid, peptide, cellulose, amino acid, sugar (for example glucose, lactose or mannose derivatives), or oligonucleotide moiety;

for each occurrence of the bracketed structure n, R₁₀ independently represents an optionally substituted C1-20 alkylenyl moiety, wherein the C1-20 alkylenyl moiety may bear one or more substituents selected from halogen or —OR where R may represent H or C1-6alkyl;

for each occurrence of the bracketed structure n, R₁₁ and R₁₂ independently represent H, an optionally substituted C1-20 alkyl, C1-20alkenyl or C1-20alkynyl moiety, an optionally substituted C1-20heteroalkyl moiety, or an optionally substituted phenyl moiety, wherein each of the foregoing C1-20 alkyl, C1-20alkenyl, C1-20alkynyl or C1-20heteroalkyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;

for each occurrence of the bracketed structure n, X independently represents an optionally substituted C1-20 alkylenyl moiety, wherein the C1-20 alkylenyl moiety may bear one or more substituents selected from halogen or —OR where R may represent H or C1-6alkyl.

In formula (I), it is to be understood that the n bracketed structures may be the same or different.

Advantageously, the hydrogel polymer may be composed of a succession of repeat units of formula I (no other monomer is used to make up the hydrogel polymer structure).

Advantageously, R¹⁰ may independently represent CH or CH—CH₂, preferably CH.

Advantageously, the hyaluronic acid, alginic acid, peptide, cellulose, amino acid, sugar (for example glucose, lactose or mannose derivatives), or oligonucleotide moiety may be incorporated via an amino group (NH₂) naturally present on the hyaluronic acid, alginic acid, peptide, cellulose, amino acid, sugar, or oligonucleotide moiety. Alternatively, the hyaluronic acid, alginic acid, peptide, cellulose, amino acid, sugar, or oligonucleotide moiety may be chemically modified to bear an amino group, prior to incorporation in the hydrogel polymer structure, as variable Y.

Advantageously, at least one occurrence of Y in the hydrogel polymer bears or comprises an organosilica particle (organosilica nanoparticle or core-shell nanocapsule, wherein the organosilica matrix may be porous (preferably mesoporous) and may contain responsively cleavable bonds within the organosilica framework (in other words, the organosilica nanoparticle or core-shell nanocapsule may be degradable upon application of an external stimulus, or may be non-degradable)), as further described infra. Advantageously, at least a subset of occurrences of Y in the hydrogel polymer bears or comprises an organosilica particle, as defined immediately above. Preferably, the organosilica particles may be functionalized so as to allow crosslinking between the hydrogel polymers (in other words, the organosilica particles allow connecting at least a monomer of formula (I) in the framework to at least another monomer of formula (I) in another framework).

Advantageously, the hydrogel polymer may be terminated by appropriate termination groups, as dictated by the chemical synthesis and reaction conditions used. For example, the hydrogel polymer may be terminated independently at each end with H, or a starting material used in the synthesis (one of the building blocks used to make up the monomer of formula (I)).

Advantageously, n, the number of monomers (I), can be such that the mass of said hydrogel polymer may be greater than about 100 kilodaltons. The number of monomers, “n”, can be such that the mass of the hydrogel polymer of formula (I) is less than about 1000 daltons. Advantageously, the mass of the hydrogel polymer of formula (I) may range from about 300 daltons to infinite, for example from about 500 daltons to infinite. The molecular mass of the hydrogel can be considered to be infinite, on account that the hydrogel network may be completely crosslinked.

Advantageously, n may be an integer between 2 and 10000, for example between 2 and 1000, between 4 and 100, between 10 and 100, between 4 and 50, preferably between 2 and 10.

Advantageously R₁₀ may independently represent a C1-20 alkylenyl moiety, for example a C1-6 alkylenyl moiety, for example —CH₂— or —CH₂—CH₂—, advantageously —CH₂—. R¹¹ and R¹² may independently represent H or C1-C6 alkyl.

Advantageously R₁₁ and R₁₂ may independently represent H, a C1-20 alkyl, C1-20alkenyl or C1-20alkynyl moiety, a C1-20heteroalkyl-moiety, or a phenyl moiety. Advantageously R₁₁ and R₁₂ may independently represent H or C1-C6 alkyl. Advantageously R₁₁ and R₁₂ may be identical. Advantageously R₁₁ and R₁₂ may represent H.

Advantageously, X may independently represent a C1-20 alkylenyl moiety, for example a C1-6 alkylenyl moiety, for example —CH₂— or —CH₂—CH₂—, advantageously —CH₂—.

Advantageously, in the linker *—R¹-L₁-R²—*, each occurrence of R¹ and R² may be identical.

Advantageously, in the linker *—R¹-L₁-R²—*, R¹ and R² may independently represent —CH₂—, —(CH₂)₂—, —(CH₂)₃—, —(CH₂)₄—, or phenyl.

Advantageously, R¹ and R² may be identical and may each represent —CH₂—, —(CH₂)₂—(CH₂)₃—, —(CH₂)₄—, or phenyl.

Advantageously, when *—R¹-L₁-R²—* represents a responsively cleavable moiety, the substituent(s) on R¹ and R² may be suitably selected to facilitate the cleavage of the responsively cleavable linker L₁ when an external signal/stimulus is applied (e.g., a change in pH (either an increase or decrease), a change in redox potential, the presence of reduction or oxidation agent, the presence of UV light or near infrared light, an enzymatic cleavage, a change in temperature, etc.). For example, the substituent(s) on R¹ and R² may be selected based on their electron-withdrawing or -donating properties, to facilitate the cleavage of the linker moiety. For example, for illustrative purposes, when L may be an imine bond and R₁ and/or R₂ may be a phenyl group, the phenyl group may bear a nitro group to make the imine bond more reactive (i.e., more responsive to cleavage upon application of a suitable stimulus).

Advantageously, L₁ may represent independently a responsively cleavable covalent bond selected from:

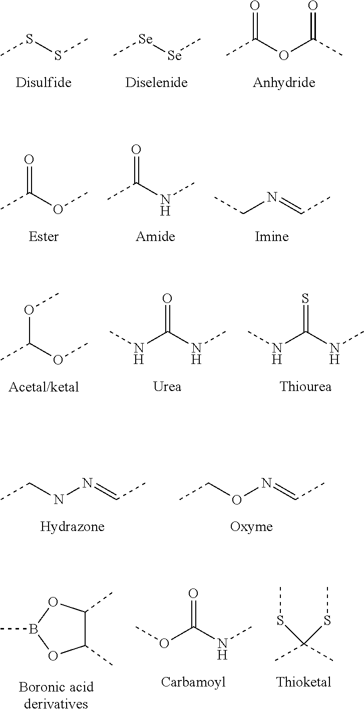

Advantageously, L₁ may independently represent or comprise a disulfide, ester, imine or hydrazone bond, preferably a disulfide bond.

Advantageously, when L₁ represents an imine bond, *—R¹-L₁-R²—* may preferably be a di-imine linker conjugated with an aromatic group such as phenyl. More preferably, *—R¹-L₁-R²—* may comprise a para di-imino phenyl moiety. Such di-imine linkers may be cleaved in acidic conditions (e.g., at pH 5-6 for 24 hours, for example pH=5.2).

Advantageously, *—R¹-L₁-R²—* may independently comprise sugar derivatives such as mannose, hyaluronic acid derivatives, collagene, aminoacids or peptides; all of which may serve as degradable crosslinker.

Advantageously, *—R¹-L₁-R²—* may represent independently a responsively pH cleavable moiety of formula (III):

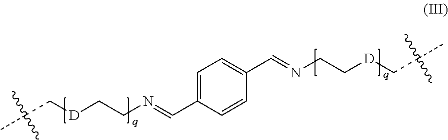

wherein each occurrence of q independently represents an integer, for example q may be an integer from 1 to 6,

D independently represents for each occurrence a C1-C3 alkylenyl moiety, or —N(Rz)- wherein Rz represents H or C1-6alkyl. As such, *—R¹-L₁-R²—* may contain more than one responsively cleavable covalent bond. In this case (linker of formula (III)), *—R¹-L₁-R²—* contains two responsively pH cleavable covalent bond (two imine bonds). Advantageously, the responsively pH cleavable moiety of formula (III) may be bound on either side to a monomer of formula (I) via a nitrogen atom (in other words, Y may be a molecular crosslinker having the structure

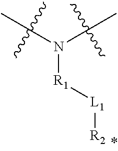

where —R¹-L₁-R²—* may have formula (III) as defined above, and * denotes the point of attachment of the molecular crosslinker to another monomer of formula (I) in the hydrogel polymer network.

Advantageously, *—R¹-L₁-R²—* may represent independently a responsively pH cleavable moiety of formula IIIa, IIIa′ or IIIb:

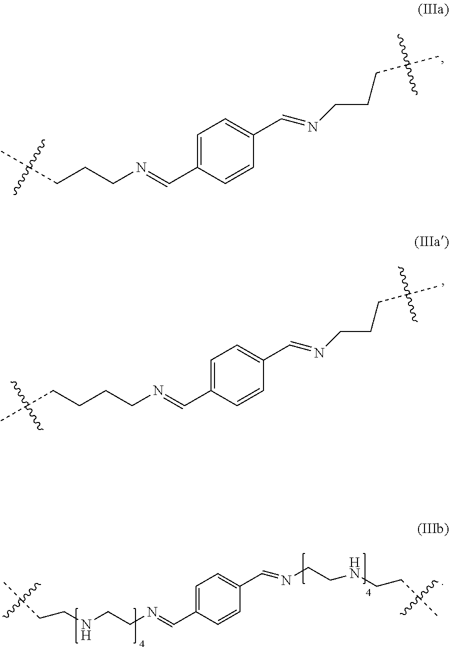

Advantageously, the responsively pH cleavable moiety of formula (IIIa), (IIIa′) or (IIIb) may be bound on either side to a monomer of formula (I) via a nitrogen atom (in other words, Y may be a molecular crosslinker having the structure

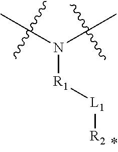

where —R¹-L₁-R²—* may have formula (IIIa), (IIIa′) or (IIIb) as defined above, and * denotes the point of attachment of the molecular crosslinker to another monomer of formula (I) in the hydrogel polymer network.

Advantageously, L₁ or —R¹-L₁-R²—* may represent independently a light responsively cleavable group and/or a photo-responsive cleavable group. The light-responsively cleavable group and/or photo-responsive cleavable group may be any suitable light responsively cleavable group and/or photo-responsive cleavable group known from the person of ordinary skill in the art. For example, —R¹-L₁-R²—* may represent a light-induced cleavable linker having formula:

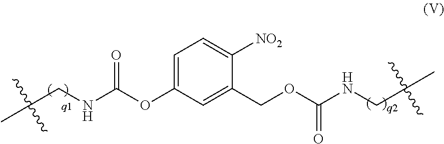

wherein q1 and q2 independently represent an integer from 1 to 6, preferably from 1 to 3. For example, q1 and q2 may both represent an integer from 1 to 6, preferably from 1 to 3, more preferably q1=q2=3. The light-sensitive linker (V) may be cleaved by irradiation with light produced by a Hg lamp. Likewise, advantageously, the light-sensitive cleavable moiety of formula (V) may be bound on either side to a monomer of formula (I) via a nitrogen atom (in other words, Y may be a molecular crosslinker having the structure

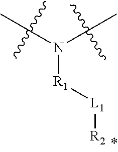

where —R¹-L₁-R²—* may have formula (V) as defined above, and * denotes the point of attachment of the molecular crosslinker to another monomer of formula (I) in the hydrogel polymer network.

Advantageously, *—R¹-L₁-R²—* may represent independently a responsively cleavable moiety selected from:

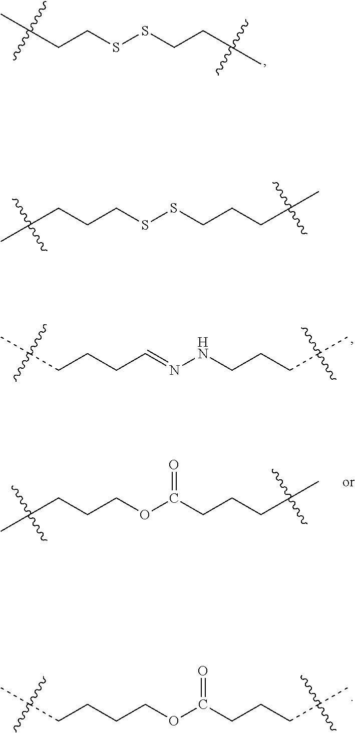

Likewise, these linkers may be bound on either side to a monomer of formula (I) via a nitrogen atom, as described above for crosslinkers III, IIIa, (IIIa′) and IIIb.

Advantageously, L₁ and *—R¹-L₁-R²—* may independently be a stable covalent bond or moiety, respectively (i.e., which is not cleaved under the conditions in which it is used/intended), for example it may be any stable bond or moiety known to the person of ordinary skill in the art and adapted to cross-link monomer and/or polymer frameworks. It may be for example a C1-20 alkylenyl moiety or C1-20 heteroalkylenyl moiety, for example a C1-6 alkylenyl or C1-6 heteroalkylenyl moiety, polyglycols, or lipids. When L₁ and *—R¹-L₁-R²—* represent a stable covalent bond or moiety, for each iteration of the monomer (I), the hydrogel is said to be non-degradable. For example, *—R¹-L₁-R²—* may represent:

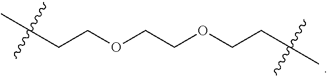 [SMILES: *CCOCCOCC*]

Advantageously, in the group of formula *—R₇(R₈)—*, R⁷ may be N and R⁸ may represent an optionally substituted C1-20 alkyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety, wherein each of the foregoing C1-20 alkyl, C1-20heteroalkyl or C1-20alkylphenyl moieties may bear one or more substituents selected from halogen, —OR, —CO₂R or —N(Rp)2 where R may represent H or C1-6alkyl, and each occurrence of Rp may independently represent H or C1-6alkyl; and the phenyl moiety may bear one or more substituents independently selected from halogen, C1-6alkyl, —NO₂, —CN, isocyano, —ORp, —N(Rp)2 wherein each occurrence of Rp independently represents H, C1-6alkyl or C₁₋₆ alkoxy.

Advantageously, Y may represent a group of formula *—N(R⁸)—*, wherein R⁸ may represent the residue of the corresponding amino acid H₂NR⁸. For example, gamma-aminobutyric acid may be used, and Y may represent *—N[(CH₂)₃CO₂H]—*.

Advantageously, in the group of formula *—R₇(R₈)—*, R⁷ may be N and R⁸ may represent a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl.

Advantageously, in the group of formula *—R₇(R₈)—*, R⁸ may represent a C1-C6 alkyl substituted with —N(Rp)2 wherein each occurrence of Rp independently represents a C1-6alkyl; for example a C1-C2 alkyl substituted with —N(Rp)2 wherein each occurrence of Rp independently represents a C1-2alkyl. R⁸ may represent a C2 alkyl substituted with —N(Rp)2 wherein each occurrence of Rp independently represents a C1alkyl. For example R⁸ mar represent —(CH₂)NMe₂.

Advantageously, in the group of formula *—R₇(R₈)—*, R⁷ may be N and R⁸ may represent R⁸ may represent a C2 alkyl substituted with —N(Rp)2 wherein each occurrence of Rp independently represents a C1alkyl. For example R⁸ mar represent —(CH₂)NMe₂.

Advantageously, in the group of formula *—R₇(R₈)—*, R⁷ may be may be N, and R⁸ may represent independently from other occurrences of R⁸ a C1-20alkylphenyl moiety optionally substituted with one or more —OR wherein R may represent H or C1-6alkyl. For example, R⁸ may represent independently from other occurrences of R⁸ a C1-6alkylphenyl moiety optionally substituted with one or more —OR wherein R may represent H or C1-6alkyl. For example, R⁸ may represent independently from other occurrences of R⁸ a C1-6alkyl moiety bearing a catechol moiety.

Advantageously, in the group of formula *—R₇(R₈)—*, R⁷ may be may be N, and R⁸ may be independently a group of following formula:

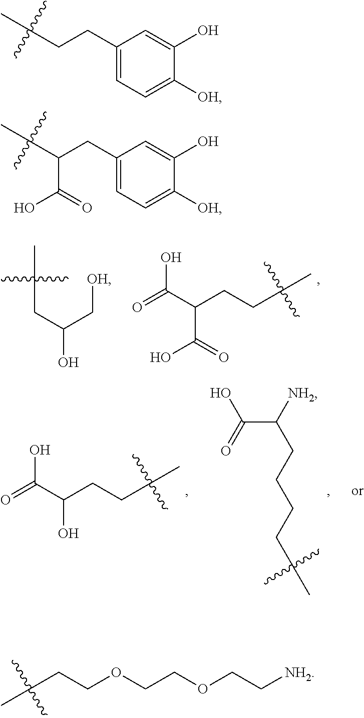

Advantageously, the hydrogels of the invention may carry biologicals molecules. In particular, Y may advantageously represent a moiety selected from hyaluronic acid, alginic acid, amino acid, peptide, cellulose, sugar (for example glucose, lactose or mannose derivatives) and oligonucleotide moieties.

Advantageously, the hyaluronic acid derivatives may be any hyaluronic acid derivatives known to the person of ordinary skill in the art. It may be for example any commercially available hyaluronic acid derivatives, for example a hyaluronic acid derivative disclosed in Voigt J et al. “Hyaluronic acid derivatives and their healing effect on bums, epithelial surgical wounds, and chronic wounds: a systematic review and meta-analysis of randomized controlled trials.” Wound Repair Regen. 2012 May-June; 20(3):317-31 [30]. For example, a hyaluronic acid moiety may be introduced in monomers of formula (I) as Y=*—N(R₈)—*, via a hydrolysed version of the naturally occurring hyaluronic acid molecule (e.g., hydrolysis of —NHAc moiety into —NH₂).

Advantageously, the alginic acid derivatives may be any alginic acid derivatives known to the person of ordinary skill in the art. It may be, for example, commercially available alginic acid or alginic acid sodium salt, from different sources and of any available molecular weight, such as alginic acid sodium salt derived from brown algae, including Laminaria hyperborea, Laminaria digitata, Laminaria japonica, Ascophyllum nodosum, and Macrocystis pyrifera, or obtained from genetic engineered bacteria. It may be chemically modified to improve adhesion or biocompatibility, for example through oxidation, functionalization or conjugation with small molecules, for example Dodecylamine, or with biomolecules, such as peptides, cellulose or sugars, as disclosed for example in K. J. Lee, D. J. Mooney, “Alginate: properties and biomedical applications”, Prog Polym Sci., 2012 January; 37(1) 106-126. [31]

Advantageously, when Y represents an amino acid it may be any amino acid known to the person of ordinary skill in the art. It may be for example D or L amino acid. It may be for example amino acid selected from the group comprising alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. It may also be gamma aminobutyric acid.

Advantageously, when Y represents a peptide moiety, it may be peptide moiety comprising for 3 to 20 amino acids, for example 3 to 5 amino acids.

Advantageously, when Y represents a sugar moiety (carbohydrate moiety), it may be any sugar known to the person of ordinary skill in the art and adapted to be linked to a polymer framework. It may be for example a sugar selected from the group comprising Arabinose, Fructose, Galactose, Glucose, Lactose, Inositol, Mannose, Ribose, Trehalose and Xylose, preferably glucose, lactose or mannose. Advantageously, these sugars may be functionalized with an amino-containing moiety, for proper incorporation of the sugar moiety as Y into the monomer of formula (I).

Advantageously, when Y represents an oligonucleotide moiety it may be derived from any oligonucleotide known to the person of ordinary skill in the art and adapted to be linked to a polymer framework. It may be for example an oligonucleotide moiety comprising from 2 to 25 Deoxyribonucleic acid and/or Ribonucleic acid. Advantageously, the oligonucleotide moiety may be functionalized with an amino-containing moiety, for proper incorporation of the oligonucleotide moiety as Y into the monomer of formula (I).

The hydrogels according to the invention may be advantageously functionalized, for example with organosilica material for example in the form of particles (organosilica nanoparticles or core-shell nanocapsules), wherein the organosilica matrix may be porous (preferably mesoporous) and may contain responsively cleavable bonds L₂ or responsively cleavable linkers #—R³-L₂-R⁴-# within the organosilica framework (in other words, the organosilica nanoparticles or core-shell nanocapsules may be degradable upon application of an external stimulus, or may be non-degradable)), as further described infra.

Advantageously, at least a subset of occurrences of Y in the hydrogel polymer may represent *—N(R⁸)—* wherein R⁸ represents a C1-20alkyl or C1-20heteroalkyl moiety, preferably C1-6alkyl or C1-6heteroalkyl, most preferably C1-6alkyl, bearing an organosilica nanoparticle, preferably the organosilica matrix may be porous, most preferably mesoporous, and may contain responsively cleavable bonds L₂ or responsively cleavable linkers #—R³-L₂-R⁴-# within the organosilica framework (R₃, R₄ and L₂ are as defined below). Advantageously, when R⁸ comprises an organosilica particle, preferably an organosilica nanoparticle, it may be bound on either side to a monomer of formula (I) via a nitrogen atom (in other words, Y may be a molecular crosslinker having the structure

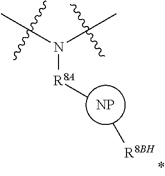

wherein R^{8A} and R^{8B} independently represent a C1-10alkyl or C1-10heteroalkyl moiety, preferably C1-6alkyl or C1-6heteroalkyl; NP denotes an organosilica nanoparticle; and * denotes the point of attachment of the molecular crosslinker to another monomer of formula (I) in the hydrogel polymer network).

Organosilica materials are well known, as well as method for preparing them, such as sol gel chemistry-based methods. The organosilica material, optionally in the form of nanoparticles, may preferably be degradable as described in WO 2015/107087, the entire contents of which are hereby incorporated by reference herein. The reader may refer to the teachings of this document for guidance as to how to prepare such degradable/disintegratable organosilica materials.

Advantageously, at least a subset of occurrences of Y in the hydrogel polymer may represent *—N(R⁸)—* wherein R⁸ represents a C1-20alkyl or C1-20heteroalkyl moiety, preferably C1-6alkyl or C1-6heteroalkyl, most preferably C1-6alkyl, bearing an organosilica core/shell nanocapsule, preferably the organosilica matrix may be porous, most preferably mesoporous, and may contain responsively cleavable bonds L₂ or responsively cleavable linkers #—R³-L₂-R⁴-# within the organosilica framework (R₃, R₄ and L₂ are as defined below). Preferably, the organosilica core/shell nanocapsule may be degradable/disintegratable in that its shell framework contains Si adjacent sites covalently bound via a responsively cleavable linker, as described in WO 2015/189402, the entire contents of which are hereby incorporated by reference herein. Advantageously, the organosilica core/shell nanocapsule may encapsulate a bioactive macromolecule or bioactive macromolecule cluster, and/or another molecule of interest that may or may not have biological activity and/or pharmaceutical or cosmetic activity. Advantageously, the bioactive macromolecule or bioactive macromolecule cluster encapsulated within the nanocapsule may be in active conformation (i.e., in a biologically active form). The reader may refer to the teachings of WO 2015/189402 for guidance as to how to prepare such degradable/disintegratable organosilica nanocapsules.

Briefly, such nanocapsules may be prepared by a method comprising steps of:
I. Producing a water-in-oil emulsion from (i) a solution of a suitable surfactant and alcohol in a suitable organic solvent, and (ii) an aqueous solution of a bioactive macromolecule or bioactive macromolecule clusters and/or another molecule of interest, a silane precursor Si(Z^{A})₄ and a selected precursor having the structure (Z)₃Si—R³-L2-R⁴—Si(Z)₃;
II. Stirring the water-in-oil emulsion obtained in step I) under alkaline conditions; thereby coating the bioactive macromolecule or bioactive macromolecule clusters with an organosilica sol-gel mixture obtained by hydrolysis-condensation of silicon alkoxide; and
III. Adding a suitable organic solvent, thereby precipitating the nanoencapsulated bioactive macromolecules or bioactive macromolecule clusters and/or other molecule of interest;
   wherein
each occurrence of Z and Z^{A} independently represents a hydrolysable or nonhydrolyzable group, provided that on each occurrence of Si of the precursor (Z)₃Si—R³-L2-R⁴—Si(Z)₃, at least one occurrence of Z represents a hydrolysable group, and at least two occurrences of Z^{A} in the precursor Si(Z^{A})₄ independently represent a hydrolysable group; wherein (i) when Z or Z^{A} represents a nonhydrolyzable group, it may be selected from an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety, wherein the substituents on the phenyl, alkyl, alkenyl, alkynyl, heteroalkyl, heteroalkenyl and heteroalkynyl moieties may be independently selected from halogen, —NO₂, —CN, isocyano, C1-6alkoxy, an oxirane/epoxyde moiety, —N(R)₂ wherein each occurrence of R is independently selected from H or C₁₋₆alkyl; and (ii) when X or X^{A} represents a hydrolysable group, it may be selected from a C1-6alkoxy, C1-6acyloxy, halogen or amino moiety; and
   R₃, R₄, L₂ and #, are as defined generally and in any variants herein.

Likewise, advantageously, when R⁸ comprises an organosilica core/shell nanocapsule, it may be bound on either side to a monomer of formula (I) via a nitrogen atom (in other words, Y may be a molecular crosslinker having the structure

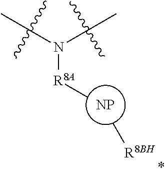

wherein R^{8A} and R^{8B} independently represent a C1-10alkyl or C1-10heteroalkyl moiety, preferably C1-6alkyl or C1-6heteroalkyl; NP denotes an organosilica core/shell nanocapsule; and * denotes the point of attachment of the molecular crosslinker to another monomer of formula (I) in the hydrogel polymer network).

Advantageously, the aforementioned organosilica material for example in the form of particles (organosilica nanoparticles or core-shell nanocapsules), may be chemically modified to bear amino-containing tether groups at the outer surface, prior to incorporation in the hydrogel polymer structure, as variable Y (cf. crosslinker *—R^{8A}—NP—R^{8B}—* mentioned above). Such functionalization may be effected by any suitable ways known in the art.

For example, such functionalization may be carried out by reacting organosilica material for example in the form of particles (e.g., organosilica nanoparticles or core-shell nanocapsules), with a silylated starting material (W)₃Si—R⁸—N(Rp)₂; each occurrence of W independently represents a hydrolysable group selected from a C1-6 alkoxy, C1-6 acyloxy, halogen or an amino moiety; R⁸ represents an optionally substituted C1-20 alkyl, C2-20 alkenyl or C2-20 alkynyl moiety, an optionally substituted C1-20 heteroalkyl, C2-20 heteroalkynyl or C2-20 heteroalkynyl moiety, or an optionally substituted phenyl moiety, wherein the substituents on the phenyl, alkyl, alkenyl, alkynyl, heteroalkyl, heteroalkenyl and heteroalkynyl moieties may be independently selected from halogen, —NO₂, —CN, isocyano, C1-6 alkoxy, an oxirane/epoxyde moiety, —N(R)₂ wherein each occurrence of R is independently selected from H or C1-6 alkyl; and each occurrence of Rp independently represents H or C1-6 alkyl. Advantageously, each occurrence of W may independently represent Cl, —OMe, —OEt, -OiPr or -OtBu. Advantageously, R⁸ may represent a C1-20alkyl or C1-20heteroalkyl moiety, preferably C1-6alkyl or C1-6heteroalkyl, most preferably C1-6alkyl.

Alternatively, such functionalization may be carried out by preparing the organosilica material for example in the form of particles (organosilica nanoparticles or core-shell nanocapsules), with a silylated starting material (W)₃Si—R⁸—N(Rp)₂ as defined above, under conventional sol gel chemistry conditions.

As a result, organosilica material for example in the form of particles (organosilica nanoparticles or core-shell nanocapsules) bearing —R⁸—N(Rp)₂ tethers at the outer surface will be obtained, and may be used for incorporation in a subset of monomers of formula (I) of the inventive hydrogels of the invention, as variable Y (cf. crosslinker *—R^{8A}—NP—R^{8B}—* mentioned above). If at least one occurrence of Rp differs from H, the —R⁸—N(Rp)₂ tethers may be first deprotected to yield —R⁸—NH₂ tethers prior to proceeding with the functionalization of the organosilica material.

Advantageously, at least a subset of occurrences of Y may further comprise a core/shell nanocapsule, advantageously an organosilica core/shell nanocapsule, preferably the shell organosilica matrix may be porous, most preferably mesoporous, preferably the shell matrix may additionally degradable/disintegratable, with a bioactive macromolecule or bioactive macromolecule cluster encapsulated within said nanocapsule. The nanocapsule may alternatively or additionally contain another molecule of interest that may or may not have biological activity and/or pharmaceutical or cosmetic activity.

Advantageously, at least a subset of occurrences of Y may comprise a nanoencapsulated molecule or bioactive macromolecule or biomacromolecule cluster comprising
a. a nanocapsule, having a core/shell structure, and
b. a molecule of interest or bioactive macromolecule or bioactive macromolecule cluster encapsulated within said nanocapsule.

Advantageously, the shell of said nanocapsule may be made of hybrid organosilica material comprising a three-dimensional framework of Si—O bonds, wherein at least a subset of Si atoms in the material's framework are connected to at least another Si atom in the framework through a linker having the following structure:

#—R³-L₂-R⁴-#;

wherein:
each occurrence of # denotes a point of attachment to a Si atom in the hybrid organosilica material's framework;
L₂ represents a responsively cleavable covalent bond or a stable bridging ligand; preferably a responsively cleavable covalent bond; and
R³ and R⁴ independently represent an optionally substituted C1-20 alkylenyl moiety, an optionally substituted C1-20 heteroalkylenyl moiety, an optionally substituted ethenylenyl moiety, —C≡C— or an optionally substituted phenyl moiety, wherein the C1-20alkylenyl, C1-20 heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —OR^{p}, —N(R^{p})₂ wherein each occurrence of R^{p} independently represents H or C1-6alkyl.

It is to be understood that the definition of R₃, R₄, L₂ and # above, and variants detailed below, also apply to the organosilica matrix making up the organosilica nanoparticles mentioned before (plain nanoparticles), that may be incorporated into the hydrogel framework as Y variable.

Advantageously, L₂ may be any moiety that contains a responsively cleavable covalent bond, which can be cleaved upon exposure to a determined stimulus. Advantageously, L₂ may represent a responsively cleavable covalent bond selected from:

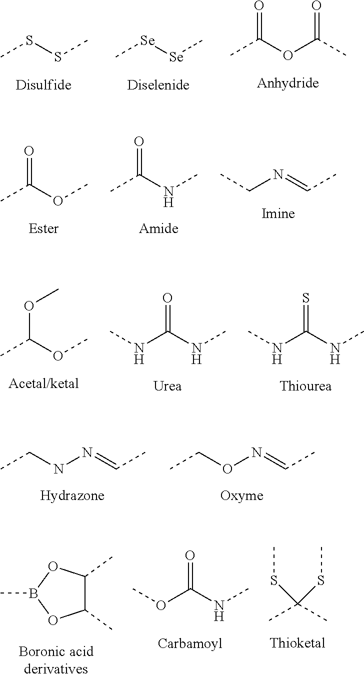

Advantageously, when L₂ represents an imine bond, #—R³-L₂-R⁴-# may preferably be a di-imine linker conjugated with an aromatic group such as phenyl. More preferably, #—R³-L₂-R⁴-# may comprise a para di-imino phenyl moiety. As discussed above, such di-imine linkers may be cleaved in acidic conditions (e.g., at pH 5-6 for 24 hours, for example pH=5.2) thereby leading to disintegration of the organosilica particles.
Advantageously, #—R³-L₂-R⁴-# may represent independently a responsively pH cleavable moiety of formula (III)

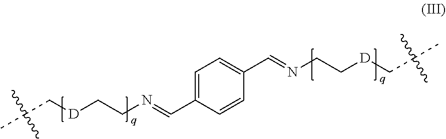

wherein q is an integer, for example q may be equal to 1 to 6,

D independently represents for each occurrence a C1-C3 alkylenyl moiety, or —N(Rz)- wherein Rz represents H or C1-6alkyl. As such, *—R³-L₂-R⁴—* may contain more than one responsively cleavable covalent bond. In this case (linker of formula (III)), *—R³-L₂-R⁴—* contains two responsively pH cleavable covalent bond (two imine bonds).

Advantageously, #—R³-L₂-R⁴-# may represent independently a responsively pH cleavable moiety of formula IIIa, IIIa′ or IIIb:

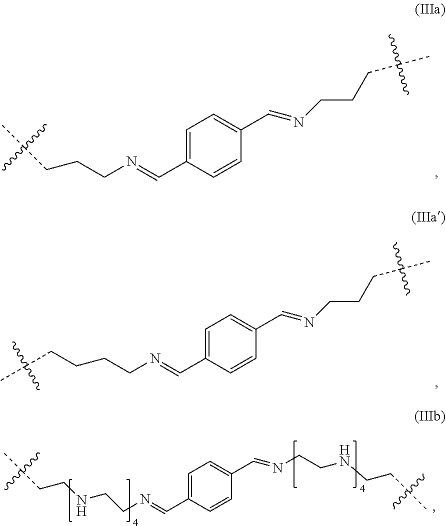

Advantageously, L₂ or #—R³-L₂-R⁴-# may represent independently a light responsively cleavable group and/or a photo-responsive cleavable group. The light-responsively cleavable group and/or photo-responsive cleavable group may be any suitable light responsively cleavable group and/or photo-responsive cleavable group known from a person of ordinary skill in the art. For example, #—R³-L₂-R⁴-# may represent a light-sensitive linker having formula:

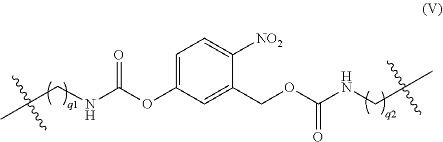

wherein q1 and q2 independently represent an integer from 1 to 6, preferably from 1 to 3. For example, q1 and q2 may both represent an integer from 1 to 6, preferably from 1 to 3, more preferably q1=q2=3. The light-sensitive linker (V) may be cleaved by irradiation with light produced by a Hg lamp.

Advantageously, #—R³-L₂-R⁴-# may represent independently a responsively cleavable moiety selected from

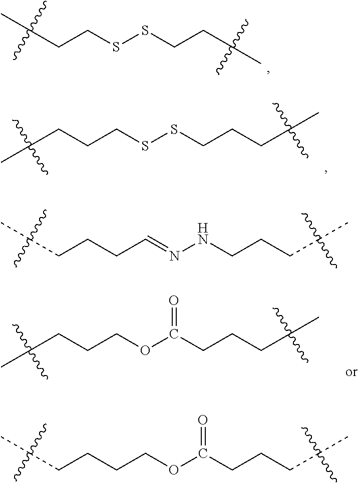

Preferably, L₂ may represent a responsively cleavable covalent bond selected from disulfide, diselenides, imine, amide, ester, urea, hydrazone or thiourea; preferably disulfide, imine (preferably #—R³-L₂-R⁴-# may comprise a para di-imino phenyl moiety), ester, or hydrazone; more preferably disulfide.

Advantageously, the bioactive macromolecule or bioactive macromolecule cluster encapsulated within the nanocapsule may be in active conformation (i.e., in a biologically active form).

Advantageously, the bioactive macromolecule or bioactive macromolecule cluster encapsulated within the nanocapsule may be in a undenatured state.

Advantageously, the bioactive macromolecule or bioactive macromolecule cluster encapsulated within the nanocapsule may remain in a folded position and retain an active conformation.

Advantageously, in the linker #—R³-L₂-R⁴-#, each occurrence of R³ and R⁴ may be identical.

Advantageously, in the linker #—R³-L₂-R⁴-#, R³ and R⁴ may be any organic radical from any commercially available silylated derivative suitable for sol-gel chemistry. For example R³ and R⁴ may independently represent-CH₂—, —(CH₂)₂—, —(CH₂)₃—, —(CH₂)₄—, or phenyl.

Advantageously, the substituent(s) on R³ and R⁴ may be suitably selected to facilitate the cleavage of the responsively cleavable linker #—R³-L₂-R⁴-# when an external signal/stimulus is applied (e.g., a change in pH (either an increase or decrease), a change in redox potential, the presence of reduction or oxidation agent, the presence of UV light or near infrared light, an enzymatic cleavage, a change in temperature, etc.). For example, the substituent(s) on R³ and R⁴ may be selected based on their electron-withdrawing or -donating properties, to facilitate the cleavage of the linker moiety. For example, for illustrative purposes, when L₂ may be an imine bond and R₃ and/or R₄ may be a phenyl group, the phenyl group may bear a nitro group to make the imine bond more reactive (i.e., more responsive to cleavage upon application of a suitable stimulus).

Advantageously the nanocapsule outer surface may comprise one or more groups of formula

#—R⁵R⁶

wherein
each occurrence of # denotes a point of attachment to a Si atom at the outer surface of the hybrid organosilica material's framework;
each occurrence of R⁵ independently represents an optionally substituted C1-20alkylenyl moiety, an optionally substituted C1-20heteroalkylenyl moiety, an optionally substituted ethenylenyl moiety, —C≡C— or an optionally substituted phenyl moiety, wherein the C1-20alkylenyl, C1-20heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO2, —CN, isocyano, —ORp, —N(Rp)2 wherein each occurrence of Rp independently represents H or C1-6alkyl; and
each occurrence of R⁶ independently represents —OR, —SR or —N(Rf)₂; preferably —N(Rf)₂; wherein each occurrence of R and Rf independently represents H or C1-6alkyl.

Advantageously, R⁵ may represent a C1-20 alkyl moiety, for example a C1-6alkyl, for example CH₂—, —(CH₂)₂—, —(CH₂)₃—, —(CH₂)₄—.

Advantageously, R⁶ represents an amino group, preferably —N(Rf)₂ wherein each occurrence of Rf independently represents H or C1-6alkyl, for example R⁶ may represent —NH₂.

Advantageously, the core-shell nanocapsules may be in the form of nanoparticles. For example, core-shell nanocapsules according to the invention may have a diameter from 1 to 999 nanometers, preferably from 1 to 500 nm, more preferably from 1 to 250 nm and most particularly from 1 to 100 nm. Advantageously, core-shell nanocapsules according to the invention may have a diameter from 25 to 500 nm, preferably from 25 to 200 nm, preferably from 40 to 90 nm, preferably from 40 to 80 nm, preferably from 50 to 70 nm.

Advantageously, the shape of the organosilica particles (filled particles or core/shell nanocapsules) may be tuned to obtain mostly particles of a specific shape (spherical, rice-shape, etc. . . . ) according to known methods. The particle shape may in turn have an effect on the mechanical properties of the hydrogel.

Advantageously, the molecule of interest may be selected from proteins, enzymes, oligonucleotides, antibodies, peptides, PNA, DNA, RNA, gene fragments and small molecules with or without pharmaceutical or cosmetic activity.

Advantageously, the proteins may be fluorescence protein family such as GFP, RFP; Cytotoxic proteins such as: TRAIL/APO-2L, Onconase, Ricin, Parasporin; Therapeutic proteins: Insulin Family, Angiopoietin family, Coagulation factor proteins, Dystrophin, HIV antigen, Hepatitis C antigen.

Advantageously, the protein may be proteins for cosmetic for example Botulinum toxin protein family, Elastin, Collagen, Keratin, Calcitonin, Silk proteins.

Advantageously, the enzymes may be RNAase, Hyaluronidase, Lysosomal enzyme acid alpha-glucosidase, Galactosidase, Glucocerebrosidase, Streptokinase, Urokinase, Altepase, Thymidine kinase, cytosine deaminase.

Advantageously, the oligonucleotides may be DNA (Deoxyribonucleic acid), RNA (Ribo Nucleic acid), PNA (Peptide Nucleic acid), LNA (Locked Nucleic Acid).

Advantageously, the antibodies may be selected from the group comprising Trastuzumab, Bevacizumab, Cetuximab, Mylotarg, Alemtuzumab, Rituximab, Brentuximab.

Advantageously, the small molecules with or without pharmaceutical activity may be for example sugars and/or polypeptide.

Advantageously, the nanoencapsulated biomolecule may be selected from proteins, enzymes, oligonucleotides, antibodies, peptides, PNA, DNA, RNA, and gene fragments.

Advantageously, the hybrid organosilica nanocapsule shell may be in the form of nanoparticles. For example, the hybrid organosilica nanocapsule shell according to the invention may have a diameter from 1 to 999 nanometers, preferably from 1 to 500 nm, more preferably from 1 to 250 nm and most particularly from 1 to 100 nm. Advantageously, the hybrid organosilica nanocapsule shell according to the invention may have a diameter from 25 to 500 nm, preferably from 25 to 200 nm, preferably from 40 to 90 nm, preferably from 40 to 80 nm.

Advantageously, the cleavage/degradation of the linker *—R¹-L₁-R₂—* and/or #—R³-L₂-R⁴-# may be independently triggered by any suitable means. For example, it may be a change in pH (either an increase or a decrease), a change in redox potential, the presence of reduction or oxidation agent, application of UV, visible or near infrared light, ultrasounds, electromagnetic radiation, a change in temperature, enzymatic cleavage, DNA binding, etc. . . . The following Table 1 gives examples of cleavage/degradation triggering means for each of the aforementioned types of responsively cleavable linkers:

TABLE 1

| | Exemplary |
|---|---|
| Linker | Triggers |
| Disulfide | Reducing |
| | agents (e.g., |
| | NaBH₄, |
| | dithiothreitol |
| | (DTT), |
| | glutathione |
| | (GSH)) |
| Diselenide | Reducing |
| | agents (e.g. |
| | thiols, metal |
| | complexes) |
| Ester | pH, |
| | enzymatic |
| | cleavage |
| | (e.g. |
| | esterase) [8] |
| Amide | Enzymatic |
| | cleavage |
| | (e.g. |
| | amidase) [9] |
| Imine | pH |
| Acetal/ketal/thioketal | pH |
| Anhydride | pH |
| Urea/thiourea | Enzymatic |
| | cleavage |
| | (e.g. urease) |
| | [10] |
| Hydrazone | pH |
| Oxyme | pH |
| Boronic acid (complexed with diols) | pH, sugars |
| Boronic esters | pH, reducing |
| | agents (e.g., |
| | LiAlH₄) |
| 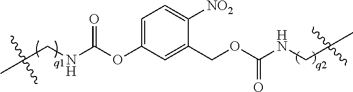 | Hg lamp irradiation |

*—R¹-L₁-R²—* and #—R³-L₂-R⁴-# may be the same or different. When they are different (especially when the type of cleavable bond(s) in the linkers is different and/or are cleaved with a different stimulus), the degradation of the hydrogel network may be controlled/effected independently from the degradation of the organosilica material (degradable nanoparticles or core/shell nanocapsules) that may be covalently bound to the hydrogel framework.

In yet another aspect, there is provided a method for producing a new class of hydrogel materials.

This new class of materials includes polymer framework systems whose framework is formed from precursors having one of the following structures:
a monomer precursor of formula (IV)

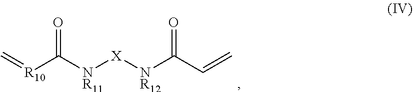

at least one bivalent molecular crosslinker precursor having the structure A-R¹-L₁-R²-A,
wherein each occurrence of A independently represents a hydrolysable or nonhydrolyzable group, provided that at least one occurrence of A represents a hydrolysable group, wherein (i) when A represents a nonhydrolyzable group, it may be selected from an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety, wherein the substituents on the phenyl, alkyl, alkenyl, alkynyl, heteroalkyl, heteroalkenyl and heteroalkynyl moieties may be independently selected from halogen, —NO₂, —CN, isocyano, C1-6alkoxy, an oxirane/epoxyde moiety, —N(R)₂ wherein each occurrence of R is independently selected from H or C1-6alkyl; and (ii) when A represents a hydrolysable group, it may be selected from a C1-6alkoxy, C1-6acyloxy, halogen or amino moiety;
L₁ independently represents a stable or responsively cleavable covalent bond; and
R¹ and R² independently represent an optionally substituted C1-20alkylenyl moiety, an optionally substituted C1-20heteroalkylenyl moiety, an optionally substituted ethylenyl moiety, —C≡C— or an optionally substituted phenyl moiety, wherein the C1-20alkylenyl, C1-20heteroalkylenyl or ethylenyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —OR^{p}, —N(R^{p})₂ wherein each occurrence of R^{p} independently represents H or C1-6alkyl,
R₁₀ independently represents an optionally substituted C1-20 alkylenyl moiety,
R₁₁ and R₁₂ independently represent H, an optionally substituted C1-20 alkyl moiety, an optionally substituted C1-20 alkylenyl moiety, an optionally substituted C1-20heteroalkylenyl moiety, an optionally substituted ethylenyl moiety, —C≡C— or an optionally substituted phenyl moiety, wherein the C1-20alkylenyl, C1-20heteroalkylenyl or ethylenyl moiety may bear one or more substituents selected from halogen or —OR where R may represent H or C1-6alkyl, and the phenyl moiety may bear one or more substituents independently selected from halogen, C1-6alkyl, —NO2, —CN, isocyano, —ORp, —N(Rp)2 wherein each occurrence of Rp independently represents H or C1-6alkyl,
X independently represents an optionally substituted C1-20 alkyl moiety.

Advantageously, in the molecular crosslinker precursor A-R¹-L₁-R²-A, each occurrence of A may independently represent a nucleophilic moiety, preferably one that can undergo a Michael-type nucleophilic addition onto the double bond of the monomer precursor (IV). For example, each occurrence of A may independently represent —N(Rf)₂ wherein each occurrence of Rf may represent H or C1-6alkyl.

Advantageously, L¹, R¹, R², R¹⁰, R¹¹, R¹² and X are independently as defined generally and in any variants above.

Thus, in one aspect, there is provided a method of preparing a hydrogel by covalently introducing a preselected precursor (general structure: monomer precursor of formula (IV)) with a molecular crosslinker precursor (general structure: A-R¹-L₁-R²-A) as defined herein, in the framework of the hydrogel material itself.

As such, the hydrogels present controlled self-destructive behavior in the environment where it is intended to perform its activity. The controlled self-destructive behavior is a property that provides numerous avenues of important applications for such hydrogel, ranging from medical to cosmetics.

The practitioner has a well-established literature of polymer and/or hydrogel materials chemistry to draw upon, in combination with the information contained herein, for guidance on synthetic strategies, protecting groups, and other materials and methods useful for the synthesis of the disintegratable materials of this invention.

Advantageously, the method may comprise steps of:
a) dissolving in water or alcoholic solutions:
   a monomer precursor of formula (IV)

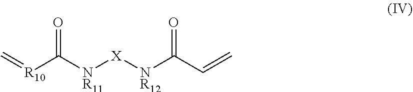

at least one bivalent molecular crosslinker precursor having the structure A-R¹-L₁-R²-A,
Optionally, disintegratable organosilica nanoparticles bearing amino-containing tether groups at the outer surface; or disintegratable organosilica core/shell nanocapsules bearing amino-containing tether groups at the outer surface and encapsulating a bioactive macromolecule or bioactive macromolecule cluster, and/or another molecule of interest that may or may not have biological activity and/or pharmaceutical or cosmetic activity; wherein the bioactive macromolecule or bioactive macromolecule cluster encapsulated within the nanocapsule is preferably in active conformation. and
Optionally, a selected precursor of formula B—R⁸
b) Stirring the solution obtained in step a), at any appropriate temperature, thereby allowing the polymerization carried out to form the hydrogel,
c) Optionally adding a suitable organic solvent, thereby precipitating the hydrogel;

wherein:
each occurrence of A or B independently represents a hydrolysable or nonhydrolyzable group, provided that at least one occurrence of A represents a hydrolysable group, wherein (i) when A or B independently represents a nonhydrolyzable group, it may be selected from an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety, wherein the substituents on the phenyl, alkyl, alkenyl, alkynyl, heteroalkyl, heteroalkenyl and heteroalkynyl moieties may be independently selected from halogen, —NO₂, —CN, isocyano, C1-6alkoxy, an oxirane/epoxyde moiety, —N(R)₂ wherein each occurrence of R is independently selected from H or C1-6alkyl; and (ii) when A or B independently represents a hydrolysable group, it may be selected from a C1-6alkoxy, C1-6acyloxy, halogen or amino moiety;
L₁ independently represents a responsively cleavable covalent bond, a moiety containing a responsively cleavable covalent bond or a stable covalent bond; and R¹ and R² independently represent an optionally substituted C1-20 alkylenyl moiety, an optionally substituted C1-20heteroalkylenyl moiety, an optionally substituted ethenylenyl moiety, —C≡C— or an optionally substituted phenyl moiety, wherein the C1-20 alkylenyl, C1-20 heteroalkylenyl or ethenylenyl moiety may bear one or more substituents selected from halogen or —OR where R may represent H or C1-6 alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl; wherein *—R¹-L₁-R²—* may independently comprise a sugar derivative such as mannose, a hyaluronic acid derivative, collagene, an amino acid or a peptide moiety;
R₁₀ independently represents an optionally substituted C1-20 alkylenyl moiety, wherein the C1-20 alkylenyl moiety may bear one or more substituents selected from halogen or —OR where R may represent H or C1-6alkyl;
R¹¹ and R₁₂ independently represent an optionally substituted C1-20 alkyl, C1-20alkenyl or C1-20alkynyl moiety, an optionally substituted C1-20heteroalkyl moiety, or an optionally substituted phenyl moiety, wherein each of the foregoing C1-20 alkyl, C1-20alkenyl, C1-20alkynyl or C1-20heteroalkyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
X independently represents an optionally substituted C1-20 alkylenyl moiety, wherein the C1-20 alkylenyl moiety may bear one or more substituents selected from halogen or —OR where R may represent H or C1-6alkyl.

Advantageously, in the molecular crosslinker precursor A-R¹-L₁-R²-A, each occurrence of A may independently represent a nucleophilic moiety, preferably one that can undergo a Michael-type nucleophilic addition onto the double bond of the monomer precursor (IV). For example, each occurrence of A may independently represent —N(Rf)₂ wherein each occurrence of Rf may represent H or C1-6alkyl.

Advantageously, at least two different molecular crosslinker precursors A-R¹-L₁-R²-A are used, wherein in one molecular crosslinker precursor L₁ represents a responsively cleavable covalent bond or a moiety containing a responsively cleavable covalent bond as described generally and in any variant herein, and in the other L₁ represents a stable covalent bond.

Advantageously, R₁, R₂, R₁₀, R₁₁, R₁₂, L₁, and X may be as described generally and in any variant above, and in any combination.

Advantageously, the monomer precursor may be of formula (IVa)

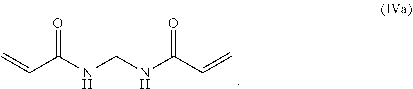

Advantageously, the amount and/or concentration of monomer precursor dissolved in solution of step a) may range anywhere from 0.1% to 100% w/v. For example, it may be from 2% to 30% w/v, for example from 4% to 30% w/v, preferably from 9% to 18% w/v.

Advantageously, the molecular crosslinker precursor A-R¹-L₁-R²-A may independently be a precursor selected from the group comprising:

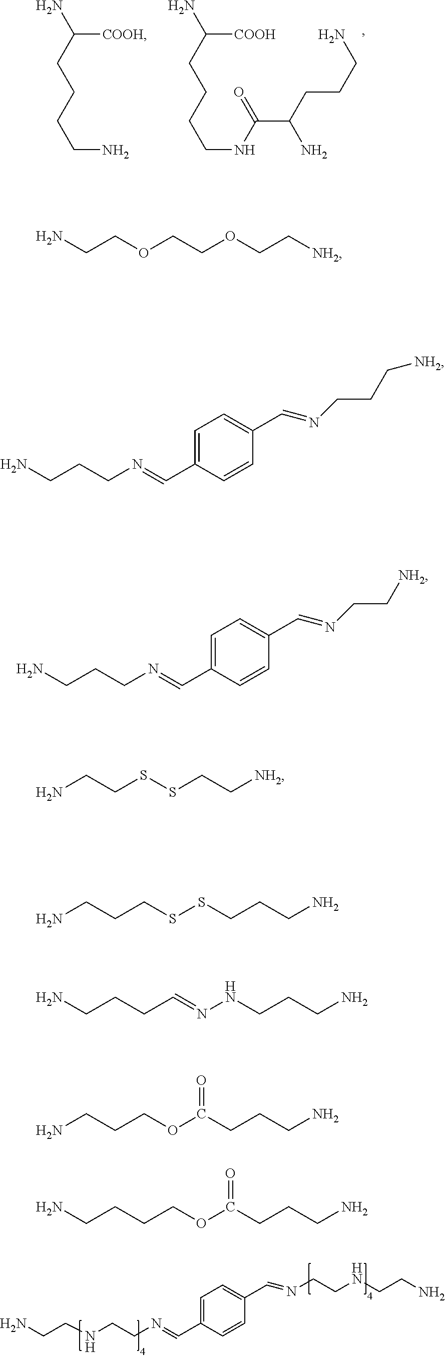

Advantageously, the amount and/or concentration of molecular crosslinker precursor dissolved in solution of step a) may range anywhere from 0.1% to 100% w/v. For example, it may be from 0.5% to 20% w/v, for example from 1% to 20% w/v, preferably from 2% to 8% w/v.

Advantageously, when the process comprises in step a) selected precursor of formula B—R⁸, B may independently represent a hydrolysable or nonhydrolyzable group, wherein (i) when B represents a nonhydrolyzable group, it may be selected from an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety, wherein the substituents on the phenyl, alkyl, alkenyl, alkynyl, heteroalkyl, heteroalkenyl and heteroalkynyl moieties may be independently selected from halogen, —NO₂, —OH, —CN, isocyano, C1-6alkoxy, an oxirane/epoxyde moiety, —N(R)₂ wherein each occurrence of R is independently selected from H or C1-6alkyl; and (ii) when B represents a hydrolysable group, it may be selected from a C1-6alkoxy, C1-6acyloxy, halogen or amino moiety. Preferably B represents N(R)₂ wherein each occurrence of R is independently selected from H or C1-6alkyl.

Advantageously, in a selected precursor of formula B—R⁸, B may independently represent a nucleophilic moiety, preferably one that can undergo a Michael-type nucleophilic addition onto the double bond of the monomer precursor (IV). For example, each occurrence of A may independently represent —N(Rf)₂ wherein each occurrence of Rf may represent H or C1-6alkyl. Advantageously, R⁸ may be as described generally and in any variant above. For example,
R⁸ may represent an optionally substituted C1-20 alkyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety, wherein each of the foregoing C1-20 alkyl, C1-20heteroalkyl or C1-20alkylphenyl moieties may bear one or more substituents selected from halogen, —OR, —CO₂R or —N(Rp)2 where R may represent H or C1-6alkyl, and each occurrence of Rp may independently represent H or C1-6alkyl; and the phenyl moiety may bear one or more substituents independently selected from halogen, C1-6alkyl, —NO2, —CN, isocyano, —ORp, —N(Rp)2 wherein each occurrence of Rp independently represents H, C1-6alkyl or C1-6 alkoxy;
R⁸ may represent the residue of the corresponding amino acid H₂NR⁸;
R⁸ may represent a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl;
R⁸ may represent a C1-C6 alkyl substituted with —N(Rp)2 wherein each occurrence of Rp independently represents a C1-6alkyl; for example a C1-C2 alkyl substituted with —N(Rp)2 wherein each occurrence of Rp independently represents a C1-2alkyl. R⁸ may represent a C2 alkyl substituted with —N(Rp)2 wherein each occurrence of Rp independently represents a C1alkyl. For example R⁸ mar represent —(CH₂)NMe₂;
R⁸ may represent a C2 alkyl substituted with —N(Rp)2 wherein each occurrence of Rp independently represents a C1alkyl. For example R⁸ mar represent —(CH₂)NMe₂;
R⁸ may represent independently from other occurrences of R8 a C1-20alkylphenyl moiety optionally substituted with one or more —OR wherein R may represent H or C1-6alkyl. For example, R⁸ may represent independently from other occurrences of R8 a C1-6alkylphenyl moiety optionally substituted with one or more —OR wherein R may represent H or C1-6alkyl. For example, R⁸ may represent independently from other occurrences of R8 a C1-6alkyl moiety bearing a catechol moiety;
R⁸ may be independently a group of following formula:

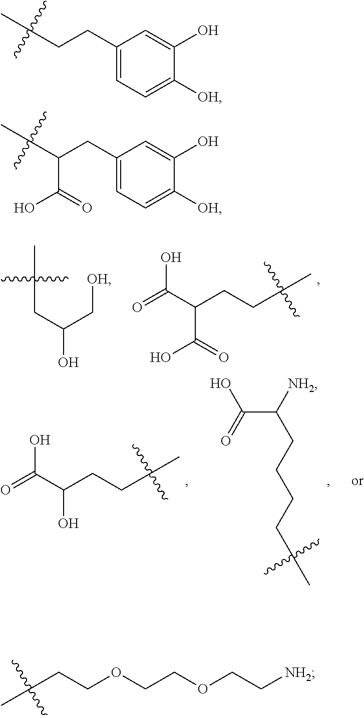

R⁸ may be independently a hyaluronic acid, alginic acid, peptide, cellulose, amino acid, sugar (for example glucose, lactose or mannose derivatives), or oligonucleotide moiety;
R⁸ may be independently a C1-20alkyl or C1-20heteroalkyl moiety, preferably C1-6alkyl or C1-6heteroalkyl, most preferably C1-6alkyl, bearing an organosilica core/shell nanocapsule, preferably the organosilica matrix may be porous, most preferably mesoporous. Preferably, the organosilica core/shell nanocapsule may be degradable/disintegratable in that its shell framework contains Si adjacent sites covalently bound via a responsively cleavable linker, as described in WO 2015/189402. Advantageously, the organosilica core/shell nanocapsule may encapsulate a bioactive macromolecule or bioactive macromolecule cluster, and/or another molecule of interest that may or may not have biological activity and/or pharmaceutical or cosmetic activity.

Advantageously, when the process comprises in step a) a selected precursor of general formula B—R⁸, it may be selected from the group comprising:

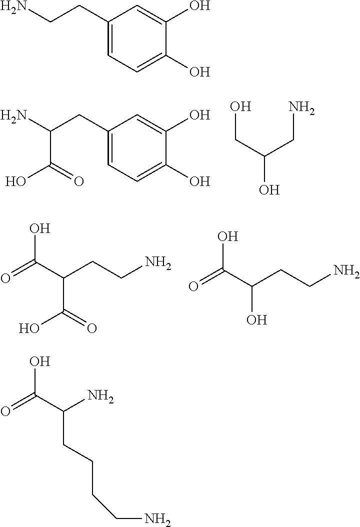

Advantageously, the amount and/or concentration of precursor (general formula B—R⁸) dissolved in solution of step a) may range anywhere from 0.1% to 100% w/v. For example, it may be from 1% to 10% w/v, preferably from 1% to 5% w/v.

Advantageously, when the process comprises in step a) the addition of nanoencapsulated molecules or bioactive macromolecules or biomacromolecule cluster, it advantageously allows to prepare hydrogels comprising nanoencapsulated molecules or bioactive macromolecules or biomacromolecule cluster.

Advantageously, the nanoencapsulated molecules or bioactive macromolecules or biomacromolecule cluster is as mentioned above, generally and in any variant described above, and in any combination.

Advantageously, the shell of said nanocapsule may be made of hybrid organosilica material comprising a three-dimensional framework of Si—O bonds, wherein at least a subset of Si atoms in the material's framework are connected to at least another Si atom in the framework through a linker having the following structure:

#—R³-L₂-R⁴-#;

wherein:
each occurrence of # denotes a point of attachment to a Si atom in the hybrid organosilica material's framework;
L₂ independently represents a responsively cleavable covalent bond or a stable bridging ligand; preferably a responsively cleavable covalent bond; and
R³ and R⁴ independently represent an optionally substituted C1-20 alkylenyl moiety, an optionally substituted C1-20 heteroalkylenyl moiety, an optionally substituted ethenylenyl moiety, —C≡C— or an optionally substituted phenyl moiety, wherein the C1-20alkylenyl, C1-20 heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —OR^{p}, —N(R^{p})₂ wherein each occurrence of R^{p} independently represents H or C1-6alkyl,
and
the nanocapsule outer surface may comprise a group of formula

#—R⁵R⁶
wherein
each occurrence of # denotes a point of attachment to a Si atom in the hybrid organosilica material's framework;
R⁵ independently represents an optionally substituted C1-20alkylenyl moiety, an optionally substituted C1-20heteroalkylenyl moiety, an optionally substituted ethenylenyl moiety, —C≡C— or an optionally substituted phenyl moiety, wherein the C1-20alkylenyl, C1-20heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO2, —CN, isocyano, —ORp, —N(Rp)2 wherein each occurrence of Rp independently represents H or C1-6alkyl;

R⁶ independently represents —OR, —SR or —N(Rf)₂; preferably —N(Rf)₂; wherein each occurrence of R and Rf independently represents H or C1-6alkyl.

Advantageously, #—R³-L₂-R⁴-# may be as defined generally and in any variant above. For example, #—R³-L₂-R⁴-# may represent independently a responsively cleavable moiety selected from:

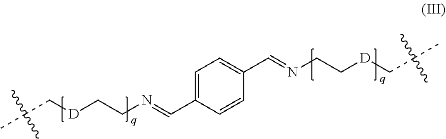

wherein q and D are as defined generally and in any variant above;

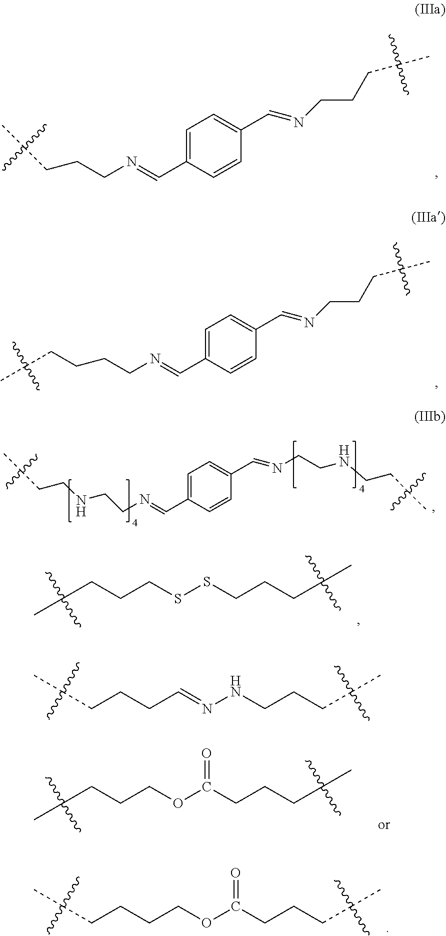

#—R³-L₂-R⁴-# may be introduced in the hybrid organosilica framework via a precursor

(Z)₃Si—R³-L2-R⁴—Si(Z)₃;

wherein L₂, R³, and R⁴ are as defined generally and any variant above, which is chemically inserted within the framework of the hybrid organosilica matrix via sol-gel chemistry. In the above, Z may independently represent a hydrolysable or nonhydrolyzable group, provided that on each occurrence of Si, at least one occurrence of Z represents a hydrolysable group.

When occurrences of Z represent a hydrolysable group, it may be selected from a C1-6 alkoxy, C1-6 acyloxy, halogen or amino moiety. Advantageously, when occurrences of Z represent a hydrolysable group, Z may represent Cl, —OMe, —OEt, -OiPr or -OtBu.

When occurrences of Z represent a nonhydrolyzable group, they may independently be selected from an optionally substituted C1-20 alkyl, C2-20 alkenyl or C2-20 alkynyl moiety, an optionally substituted C1-20 heteroalkyl, C2-20 heteroalkynyl or C2-20 heteroalkynyl moiety, or an optionally substituted phenyl moiety, wherein the substituents on the phenyl, alkyl, alkenyl, alkynyl, heteroalkyl, heteroalkenyl and heteroalkynyl moieties may be independently selected from halogen, —NO₂, —CN, isocyano, C1-6 alkoxy, an oxirane/epoxyde moiety, —N(R)₂ wherein each occurrence of R is independently selected from H or C1-6 alkyl.

Advantageously, when occurrences of Z represent a nonhydrolyzable group, Z may represent C1-6 alkyl or C2-6 alkenyl; preferably -Me, -Et or —CH═CH₂; most preferably -Me or -Et.

The insertion of the responsively cleavable linker #—R³-L₂-R⁴-# within the framework of the hybrid organosilica matrix may be performed during the synthesis of the hybrid organosilica material itself, no additional step is required, if not the preparation of the required (Z)₃Si—R³-L₂-R⁴—Si(Z)₃ precursor, which may also be carried out in situ.

Advantageously, the following may be used as (Z)₃Si—R³-L₂-R⁴—Si(Z)₃ precursor:

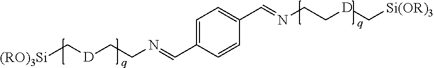

wherein q and D are as defined generally and in any variant above;

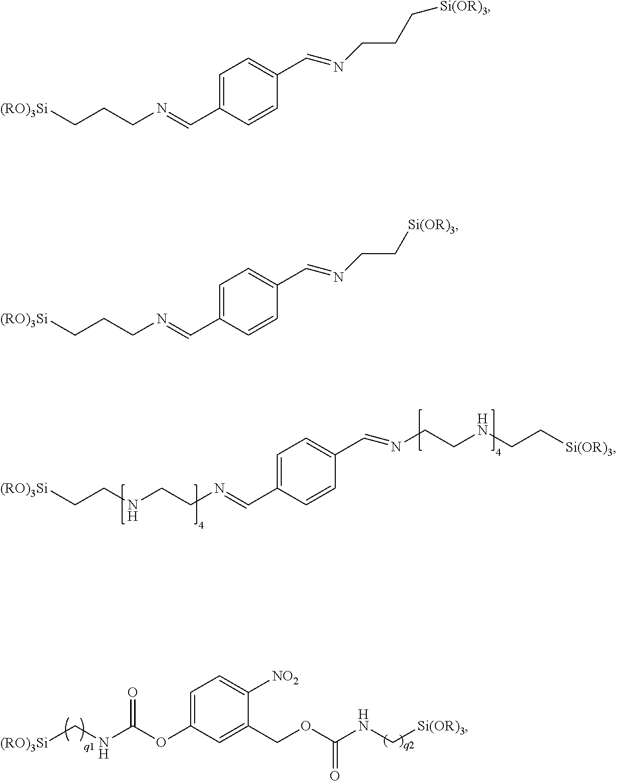

wherein q1 and q2 are as defined generally and in any variant above;

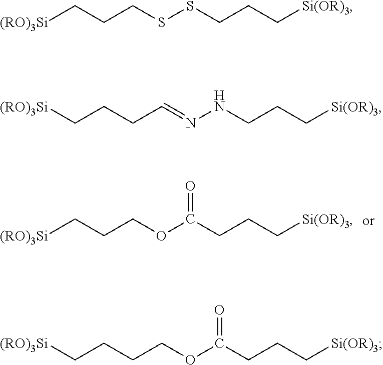

wherein each occurrence of R may independently represent Me, Et, iPr or tBu.

Advantageously, R⁵ independently represents a C1-20 alkyl moiety, for example a C1-6alkyl, for example CH₂—, —(CH₂)₂—, —(CH₂)₃—, —(CH₂)₄—.

Advantageously, R⁶ independently represents N, preferably —N(Rf)2 wherein each occurrence of Rf independently represents H or C1-6alkyl, for example —NH₂.

Advantageously, when the outer surface of the nanocapsule comprises a group of formula #—R⁵R⁶, it improves the attachment of the nanocapsule to the hydrogel framework. In particular, as illustrated herein, when a group of formula #—R⁵R⁶ as defined herein comprises an amino group, it allows to covalently link the nanocapsule to the hydrogel framework.

Functionalized organosilane chemistry is well known, and the reader may refer to the following citations for illustrative synthetic guidance that may be readily adapted in the context of the present invention. [2]

Advantageously, the amount of organosilica (nano)particles or core/shell nanocapsules with the encapsulated active molecules can vary between 0.1 and 20% w/v (weight of the silica nanoparticles vs volume of the pre-hydrogel). For example, it may be 1%, 2%, 5%, 0.1% depending on the elasticity and delivery that is desired.

Advantageously, the stirring in step b) may be carried out at any suitable temperature with any suitable process and/or device known to the person of ordinary skill in the art.

Advantageously, a pH adjusting agent may be used to modulate the pH to the desired value, for example in step b). The pH of the solution may be adjusted using any suitable technique. As the pH-adjusting agent, there can be mentioned, for example, acids such as sulfuric acid, hydrochloric acid and the like; and alkalis such as sodium hydroxide, ammonia and the like. Advantageously, when disintegratable hybrid organosilica nanoparticles or core/shell nanocapsules are used, the pH of the reaction system may be preferably adjusted to >7, for example 7.5-10, more preferably 8-9, most preferably about 8.

Advantageously, the organic solvent in step c) may be any suitable organic solvent known to the person of ordinary skill in the art. It may be for example an organic solvent selected from the group comprising methanol, ethanol, n-propanol and/or any other protic solvent, or mixture of two or more thereof.

Advantageously, the hydrogel comprising or not the nanoencapsulated bioactive macromolecule or bioactive macromolecule cluster and/or another molecule of interest that may or may not have biological activity and/or pharmaceutical or cosmetic activity, obtained with the process of the invention may be transparent.

Advantageously, the hydrogel comprising or not the nanoencapsulated bioactive macromolecule or bioactive macromolecule cluster and/or another molecule of interest that may or may not have biological activity and/or pharmaceutical or cosmetic activity, may be obtained at room temperature, for example between 20 to 35° C., in an aqueous solvent.

Advantageously, the hydrogel of the invention may be obtained according to a catalyst-free Michael-type addition.

Advantageously, the hydrogel may be formed in-situ and does not need any external agent and/or supplemental agent for the reticulation/crosslinking process.

Advantageously, the hydrogel may be formed in-situ under physiological condition.

Another object of the present invention relates to a hydrogel obtainable by a method of the invention.

Hydrogels described herein are useful for any medical application where it is desirable to fill a hole, for example a lesion, a wound, etc.

Hydrogels described herein as mentioned above are also useful for any application where controlled release of a molecule of interest, bioactive molecule or biomolecule cluster is desired.

Hydrogels described herein are also particularly adapted for uses of this type of materials where the self-destructive behavior that characterizes the core/shell silica nanocapsules and the hydrogels of the invention provides an advantage, and for applications where preservation of the biological activity of the biomacromolecule is needed.

In particular, in contrast to conventional hydrogel materials known in the art, the hydrogels described herein have the unexpected property of being formed in-situ without any external stimuli.

In addition, in contrast to conventional hydrogel materials known in the art, the hydrogels described herein allow to provide a physical support, notably for in vivo medical applications, and also be biodegradable.

Moreover, hydrogels described herein may completely lose their structural integrity (disintegration) upon application of a suitable stimuli and/or under the biological activity of proteins, for example enzymes. In other words, for biomedical applications, for example application onto tissue and/or after injection, this means less bio-accumulation, better elimination, and less toxicity.

Owing to their disintegratable properties, hydrogel comprising core/shell silica nanocapsules prove much more efficient in releasing and delivering macromolecules that they encapsulate (e.g., therapeutically and/or cosmetically active macromolecular principles). In other words, release of the macromolecules trapped/encapsulated in the core/shell silica nanocapsules occurs much more efficiently. For biomedical applications (e.g., when the framework metal is Si), this means less bio-accumulation, better elimination, and less toxicity.

Accordingly, there is provided compositions comprising hydrogel described generally and in any variants herein and any compound and/or additive suitable for any one or more of the material's intended use describe above.

For example, there is provided a pharmaceutical composition comprising hydrogel described generally and in any variants herein, and a pharmaceutically acceptable carrier, adjuvant or vehicle. In exemplary embodiments, these compositions optionally further comprise one or more additional therapeutic agents.

The person of ordinary skill in the art, taking into consideration the common technical knowledge in the medical field, would know and/or select the additional therapeutic agents in light of the disease/condition to be treated.

In another example, there is provided a cosmetic composition comprising hydrogel described generally and in any variants herein, and a cosmetically acceptable carrier, adjuvant or vehicle. In certain embodiments, these compositions optionally further comprise one or more additional cosmetically useful agents.

In yet another example, there is provided a veterinary composition comprising hydrogel described generally and in any variants herein, and a pharmaceutically acceptable carrier, adjuvant or vehicle. In exemplary embodiments, these compositions optionally further comprise one or more additional therapeutic agents.

In another aspect, there is provided a hydrogel described generally and in any variants herein, for use as medicament.

In another aspect, there is provided a hydrogel described generally and in any variants herein, for use as medicament for sealing a wound, for enhancing tissue regeneration, fillers for example for submucosal fluid cushion for surgery, tissue reconstitution in a subject-in-need thereof.

In yet another aspect, there is provided a hydrogel described generally and in any variants herein, for use as medicament for treating diabetes or spinal cord injury.

In yet another aspect, there is provided a hydrogel described generally and in any variants herein, for use as medicament for treating hernia or ulcers.

In another aspect, there is provided a hydrogel described generally and in any variants herein, in a cosmetic composition.

In another aspect, there is provided a hydrogel described generally and in any variants herein, or a cosmetic composition described generally and in any variants herein, for delivering a cosmetically bioactive macromolecule and/or a cosmetically bioactive macromolecule to the skin.

The cosmetically bioactive macromolecule may be any cosmetically bioactive macromolecule and/or a cosmetically bioactive macromolecule known in the art. It may be, for example, selected from the group comprising collagen, keratin, elastin, calcitonin, hyaluronic acid, amino acids, retinol, antioxidants, vitamins or silk proteins.

In another aspect, there is provided a hydrogel described generally and in any variants herein for use as a medicament in the treatment of cancer, preferably tumors. Specifically, hydrogels described herein may be injected under a tumor to be excised, preferably a solid tumor, thereby allowing the resection of the tumor with minimal lesion to the surrounding tissue. The person of ordinary skill in the art, taking into consideration the common technical knowledge in the medical field, would know and/or select suitable therapeutic agents that may be used in association with the hydrogel for optimizing therapeutic success of the procedure. In particular, the person of ordinary skill in the art would select which therapeutic agent should be included into the hydrogel, for example in the pores and/or core of organosilica particles (plain nanoparticles or core/shell nanoparticles) that may be embedded/covalently conjugated to the hydrogel network, as detailed supra. It may be for example any anti-cancerous drug or any suitable palliative drug appropriate for this type of surgical treatment (e.g., antiinflammatory) known from a person of ordinary skill in the art that could be linked and/or included into the hydrogel and/or encapsulated into the nanoparticles.

In another aspect, there is provided hydrogel described generally and in any variants herein, for delivering a cosmetically bioactive macromolecule to the skin. In exemplary embodiments, the cosmetically bioactive macromolecule may be collagen, keratin, elastin, calcitonin or silk proteins.

In another aspect, there is provided a method for systemically delivering a bioactive macromolecule, in a biologically active form, to a subject in need thereof, the method comprising, administering to the subject a therapeutically effective amount of a hydrogel described generally and in any variants herein. In exemplary embodiments, the bioactive macromolecule may be selected from proteins, oligonucleotides, antibodies, peptides, PNA, DNA, RNA, gene fragments, a hormone, a growth factor, a protease, an extra-cellular matrix protein, an enzyme, an infectious viral protein, an antisense oligonucleotide, a dsRNA, a ribozyme, a DNAzyme, antibiotics, antinflammatory, steroids, chemotherapeutics. In exemplary embodiments, the bioactive macromolecule may be an enzyme and said biological activity is a catalytic activity. In exemplary embodiments, the bioactive macromolecule may be a hormone and said biological activity is a ligand binding activity.

In another aspect, there is provided a unit dosage form for local delivery of a molecule to a tissue of a subject, the unit dosage form comprising, a therapeutically effective amount of a hydrogel described generally and in any variants herein or a pharmaceutical composition described generally and in any variants herein. In exemplary embodiments, the molecule may be selected from proteins, oligonucleotides, antibodies, peptides, PNA, DNA, RNA, gene fragments, a hormone, a growth factor, a protease, an extra-cellular matrix protein, an enzyme, an infectious viral protein, an antisense oligonucleotide, a dsRNA, a ribozyme and a DNAzyme.

In another aspect, there is provided a method for treating a disease in a subject in need thereof, the method comprising administering to the subject a therapeutically effective amount of hydrogel described generally and in any variants herein, thereby treating the disease in the subject.

In another aspect, there is provided a delivery system for sealing a wound, for enhancing tissue regeneration, fillers for example for submucosal fluid cushion for surgery, tissue reconstitution in a subject-in-need thereof, said system comprising hydrogel described generally and in any variants herein.

In another aspect, there is provided a method of using hydrogel described generally and in any variants herein as controlled-release agents or carriers for macromolecular drug, protein, and vaccine delivery.

In another aspect, there is provided a method of using hydrogel described generally and in any variants herein for sealing acute and/or chronic wounds and/or perforation in a subject-in-need thereof, the method comprising administering to the subject a therapeutically effective amount of a hydrogel according to the invention or a pharmaceutical composition according to the invention, thereby sealing the wound and/or perforation.

In another aspect, there is provided a method for treating a disease, preferably cancer, most preferably cancer tumor, in a subject-in-need thereof, the method comprising administering to the subject a therapeutically effective amount of a hydrogel according to the invention or a pharmaceutical composition according to the invention, thereby treating the disease in the subject. Advantageously, hydrogels described herein may be injected under a tumor to be excised, preferably a solid tumor, in an amount sufficient to substantially detach/disengage the tumor from surrounding tissue, thereby allowing the resection of the tumor with minimal lesion to the surrounding tissue.

In another aspect, there is provided a method for treating diabetes, in a subject-in-need thereof, the method comprising administering to the subject a therapeutically effective amount of a hydrogel according to the invention or a pharmaceutical composition according to the invention, thereby treating the disease in the subject. The injected hydrogel may be advantageously loaded with insulin, for example encapsulated in core-shell organosilica nanoparticles conjugated to the hydrogel, as detailed supra, for sustained release of insulin.

In another aspect, there is provided a method for treating spinal cord injury, in a subject-in-need thereof, the method comprising administering to the subject a therapeutically effective amount of a hydrogel according to the invention or a pharmaceutical composition according to the invention. Advantageously, the administration may be carried out by locally injecting the hydrogel near the site of spinal cord injury. The injected hydrogel may be advantageously loaded with any drug useful for treating spinal cord injury, such as methylprednisolone, for example encapsulated in core-shell organosilica nanoparticles conjugated to the hydrogel, as detailed supra, for sustained release of the drug.

In another aspect, there is provided a method for treating hernia or ulcers, in a subject-in-need thereof, the method comprising administering to the subject a therapeutically effective amount of a hydrogel according to the invention or a pharmaceutical composition according to the invention. Advantageously, the administration may be carried out by locally injecting the hydrogel at the site of hernia or ulcer, preferably at the hernia opening to close it. The injected hydrogel may be advantageously loaded with any drug useful for ancillary treating hernia or ulcers, such as anti-infection agents or anti-inflammatory drugs, for example encapsulated in core-shell organosilica nanoparticles conjugated to the hydrogel, as detailed supra, for sustained release of the drug.

The hydrogel according to the invention therefore can find applications in in vitro and in vivo diagnostics, therapy, in cosmetics, in drug delivery, and in any other application where a release can be envisaged or prove useful.

Advantageously, unlike previous materials obtained by photo-crosslinking, or thermal gelation, the Hydrogels described generally and in any variants herein may be advantageously formed in situ via Michael-type addition reaction under physiological conditions from mixing of the monomers in aqueous solution through the formation of amine bonds.

Advantageously, Hydrogels described generally and in any variants herein can advantageously deliver active molecules, for example during the hydrogel degradation phase, and for example potentially assisting the healing of surrounding tissue at the site of injection.

Advantageously, Hydrogels described generally and in any variants herein are preferably injectable and biodegradable.

Advantageously, hydrogels described generally and in any variants herein may undergo degradation responding to cell-secreted molecules through reductive cleavage of the linker, for example of disulfide moieties, incorporated both in the nanocapsules and in the hydrogel structures.

Advantageously, hydrogels described generally and in any variants herein may release molecules of interest, for example proteins, for example from the nanocapsules, through the degradation of the nanocapsule shell.

Advantageously, hydrogels described generally and in any variants herein show advantageously a rapid gelation when injected in vivo, and for example may afforded a long-lasting high mucosal elevation.

In a variant, silicon particles, preferably silicon nanoparticles, most preferably porous silicon nanoparticles, may be used in place of or in addition to the organosilica particles mentioned in any variant herein. The outer surface of silicon particles will oxidize to silicon oxide when exposed to water or an aqueous environment. As such, hydrogels of the invention may comprise silicone particles, preferably silicon nanoparticles, most preferably porous silicon nanoparticles, mixed in with the hydrogel matrix or covalently bound thereto much like the organosilica particles described herein.

Silicon porous particles are fully degradable and have the same role of the organosilicates systems (cf J. Mater. Chem. B, 2016, 4, 7050-7059; and Nature Materials 8, 331-336 (2009)). [32]

Porous silicon has exhibited considerable potential for biological applications owing to its biocompatibility, biodegradability, and the possible surface functionalization. For in vivo use, silicon nanoparticles provide attractive chemical alternatives to other quantum dots, which have been shown to be toxic in biological environments. In addition, silicon is a common trace element in humans and a biodegradation product of porous silicon, orthosilicic acid (Si(OH)₄), is the form predominantly absorbed by humans and is naturally found in numerous tissues. Furthermore, silicic acid administered to humans is efficiently excreted from the body through the urine. Porous silicon particles have been filled with therapeutics and they can be engineered to degrade in vivo into benign components that clear renally. Therefore porous silicon particles, in particular porous silicon nanoparticles, can replace or add as component of hybrid hydrogels according to the invention.

With respect with each of the uses and methods described above, any hydrogel described generally and in any variant herein may be used.

### BRIEF DESCRIPTION OF THE DRAWING

FIG. 1. Scheme of the synthesis and functionalization of BNCs containing disulfide moiety in the framework and loaded with Cyt-C inside the silica capsule (a); SEM image of the monodispersed functionalized BNCs, in the insert SEM picture of a naked nanoparticle (b); scheme of degradation after GSH exposure and release of Cyt-C(c).

FIG. 2. Synthesis of an hydrogel according to the invention (dPAA), embedding BNCs (a); scheme of the network (b); FTIR trace of dPAA (c); SEM showing the porosity of the nanocomposite (d).

FIG. 3: FIG. 3a represents the swelling ratio of the hydrogels incubated with 10 μM GSH solution or Phosphate Buffer Saline (PBS), showing the degradation of the network (ordinate:swelling ratio) over time (abscissa:hours). FIG. 3 b are image of scanning transmission electron microscope representing the fragmentation of the particles in presence of the reducing GSH after 72 hours (ii) and image of scanning transmission electron microscope representing the particles in presence of the reducing PBS after 72 hours (i). FIG. 3 c represents cumulative release of Cyt-C from an hydrogel according to the invention by measure of absorbance at 410 nm (ordinate) overt time in hours (abscissa)

FIG. 4. HDFa-mediated degradation as function of time of dPAA hydrogels, solid line, containing 2.5×10⁵ cells and control (acellular dPAA), dashed line (a); macroscopic pictures showing the cell-mediated degradation of the dPAA (b). Overlay images (i.e brightfield and DiD channel) of the HDFa growing onto the scaffold (24 h) and gradually transferring to the bottom well plastic as the dPAA gets degraded (48 and 96 h); scale bar is 100 μm.

FIG. 5. SEM image of the explanted tissue and hydrogel, showing collagen fibers within the hydrogel matrix (a), scale bar is 100 μm; endoscopy images of the dPAA stained with Methylene Blue formed in vivo (b) and close up (c) showing with fibrous formation within the hydrogel matrix.

Change in mucosal elevation as a function of time after the injection of NS or dPAA into a resected porcine stomach (d). Methylene blue was mixed as color agent. Height values (black bar) obtained for NS were 6.7 mm, 4.2 mm and 2.9 mm after 10 sec, 10 min and 1 h respectively; for dPAA were 8.3 mm, 6.4 mm, 5.8 mm after 10 sec, 10 min and 1 h respectively.

FIG. 6. Endoscopic views of the different steps of the ESD procedure performed using a hydrogel according to the invention (dPAA) stained with Methylene Blue. Setting of the lesion, approx. 3 cm in diameter (a); injection of the dPAA solution (b); formation of the SFC after gelation of the dPAA (c); circumferential cutting (d); complete resection with protective layer of dPAA that remained adhered to the muscolaris (e); wound left after ESD with layer of the dPAA (f).

FIG. 7. Mechanism of network degradation upon exposure to GSH, schematic representation and pictures of the hydrogel network before and after degradation. The yellow lines represent the disulfide units; the degradation of the hydrogel scaffold occurs at the disulfide cleavage site by thiol-disulfide exchange.

FIG. 8. Swelling ratio of the hydrogels comprising nanoparticles containing different cystamine amounts, incubated with 10 μM GSH solution, showing the degradation of the network (ordinate:swelling ratio) over time (abscissa:hours). Hydrogel containing 10 wt % of cystamine is displayed in green, the one with 40 wt % is in red; dPAA was added as a reference.

FIG. 9. Viability of HDFa onto the degradable nanocomposite measured with alamarBlue assay (a). The plot displays the percentage of reduced alamarBlue, which is proportional to the cell metabolic activity of the cells, as function of time. The result shows an increase in metabolic activity, thus indicates the proliferation of cells onto the scaffold. Proliferation of the cells in 3D (b); 3-channel visualization of the HDFa (c), scale bar is 100 m. Cells in (b) and (c) were stained with Vybrant DiD (grey) to facilitate the imaging.

FIG. 10. Injection of the dPAA solution stained with Methylene Blue via a surgical 23-gauge needle (a); formation of a mucosal elevation (b); gelation occurred in less than 10 minutes, achieving a solid and elastic hydrogel, adhered to the tissue.

FIG. 11. ¹H NMR spectrum of DCNS before and after light irradiation.

FIG. 12. Characterization of model spherical MSPs, illustrated in the Examples.

FIG. 13. Complete characterization of the hybrid light-sensitive spherical MSPs with light-induced cleavable linkers within the organosilica matrix, illustrated in the Examples.

FIG. 14. Schematic representation of the light-induced cleavability experiments and SEM images of the investigated organosilica particles comprising light-induced cleavable linkers within the organosilica matrix.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to a person of ordinary skill from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The present invention and its applications can be understood further by the examples that illustrate some of the embodiments by which the inventive product and medical use may be reduced to practice. It will be appreciated, however, that these examples do not limit the invention. Variations of the invention, now known or further developed, are considered to fall within the scope of the present invention as described herein and as hereinafter claimed.

### EXAMPLES

### Example: Hydrogels Comprising Particles: Synthesis and In-Vitro and In-Vivo Uses

### Materials and Methods

### Abbreviations

MBA N,N-methylenebisacrylamide
GABA gamma-aminobutiric acid
MeOH Methanol
L-DOPA
Dopamine
ENA Ethylendiamine
PEHA Pentaethylenhexamine O-(2-aminomethyl)-O′-methylpolyethylene glycol,
MPGA PM 5000
CYST Cystamine
DMENA N,N-Dimethylethylenediamine
NH2-CytC@BNPs cleavable core/shell nanocapsules
1 mg/ml NH2-MSPs Solution of NH2-MSPs as described herein

Redox-Cleavable Nanocapsules Synthesis

Triton X-100 (7.08 mL) and n-hexanol (7.20 mL) were dissolved in Cyclohexane (30 mL). Separately, 1.20 mL of a 5 mg/mL aqueous solution of Cytochrome C from equine heart were mixed with 0.16 μL of tetraethyl orthosilicate and 0.24 mL of bis[3 (triethoxysilyl)propyl]disulfide.

After shaking, this mixture was added to the former organic medium. Eventually, 200 μL of 30% ammonia aqueous solution were added and the water-oil emulsion was stirred overnight at room temperature. After that, 80 mL of pure acetone were added to precipitate the NPs and the material was recovered by means of centrifugation, washing five times with water and one with ethanol.

The procedure can be adapted for the encapsulation of different globular proteins.

Redox-Cleavable Nanocapsules Functionalization

40 mg of redox-cleavable nanocapsules are suspended in 5 mL of ethanol.

44 μL of 3-aminopropyltriethoxysilane (PM=221,37, d=0,946, 0.094 mmol) and 20 μL of triethylamine (TEA) are added to the suspension, that is stirred at R.T. for 18 hours.

The resulting NH₂-functionalized redox-cleavable nanocapsules (NPs, also designated NH2-CytC@BNPs) are then washed five times with distilled water and dried.

Redox-Responsive Degradable Hydrogel Functionalized with Silica Nanoparticles 200 mg of methylenebisacrylamide (MBA), 65 mg of cystamine hydrochloride and 70 μL of N,N-dimethylethylenediamine are mixed together with 1.0 mL of a 1 mg/mL solution of NH₂-functionalized breakable nanocapsules.

After 48 h, the hydrogel is formed.

The procedure can be modified and other NH₂-functionalized silica nanoparticles can be used, such as breakable or non-breakable mesoporous silica nanoparticles.

The experimental protocol described above was repeated with four additional different compositions of non degradable (no cleavable crosslinker in the PAAm polymer network) or degradable hydrogels (presence of cleavable crosslinker in the PAAm polymer network). The hydrogel compositions and starting materials are summarized below. Unless otherwise indicated in the table below, all hydrogels formed within 48 hours.

| Hydrogel | Hydrogel | MONOMER 1 | M1 | MONOMER 2 | | M2 |
|---|---|---|---|---|---|---|
| number | Properties | (M1) | amount | (M2) | | amount |
| 1 | Degradable | MBA (mg) | 200.00 | DMENA (μl) | | 86.00 |
| 2 | Non | MBA (mg) | 200.00 | GABA (mg) | | 66.00 |
| | Degradable | | | | | |
| 3 | Non | MBA (mg) | 200.00 | GABA (mg) | | 66.00 |
| | Degradable | | | | | |
| 4 | Non | MBA (mg) | 200.00 | GABA (mg) | | 66.00 |
| | Degradable | | | | | |
| | | | | | | S1 |
| Hydrogel | CROSSLINKER | | | Other 1 | SOLVENT | amount |
| number | 1 (CL1) | CL1 amount | OTHER 1 | amount | 1 (S1) | (mL) |
| 1 | CYST (mg) | 32.00 | NH2- | 1 mg | Water | 1.50 |
| | | | CytC@BNPs | | (mL) | |
| 2 | PEHA (μl) | 30.00 | 1 mg/ml | 1.50 | | |
| | | | NH2-MSPs | | | |
| | | | (ml) | | | |
| 3 | PEHA (μl) | 60.00 | 1 mg/ml | 1.50 | | |
| | | | NH2-MSPs | | | |
| | | | (ml) | | | |
| 4 | PEHA (μl) | 120.00 | 1 mg/ml | 1.50 | | |
| | | | NH2-MSPs | | | |
| | | | (ml) | | | |

NH2-CytC@BNPs refers to NH2-functionalized core/shell redox-cleavable nanocapsules described above.

NH2-MSPs refers to NH2-functionalized hybrid light-sensitive MSPs described in Example 1.3 below.

When the hydrogels were needed for in vitro experiments (i.e. GSH degradation and cellular viability and degradation), the obtained solution was transferred to glass vials (500 μl per vial) and allowed to react in static conditions at r.t. Glass vials with inner diameter of 8 mm were used as molds. The hydrogels were obtained after 48 hours.

Once obtained, the disk-shaped hydrogels were freeze-dried and weighted. Dried hydrogels were used to study the swelling ratio at different pH and the degradation kinetics with different concentrations of GSH. This step allowed us as well to sterilized the materials for in vitro experiments.

Sterile and ultrafiltered water was used during hydrogel preparation for in vivo tests; the synthesis was carried out in closed sterile vials and protected from bacteria contamination, the final product was assumed to be free of bacterial contamination.

### Photo-Cleavable Nanoparticles Synthesis

### 1.1 2-nitro-5-(((3-(triethoxysilyl)propyl)carbamoyl)oxy)benzyl (3-(triethoxysilyl)propyl)carbamate, (DCNS)

This photosensible molecule was synthesized by the reaction of the alcohol groups of the 5-hydroxy-2-nitrophenyl alcohol and Iscocyanopropyltriethoxysilane by the presence of triethylamine as catalyst (see scheme 1)

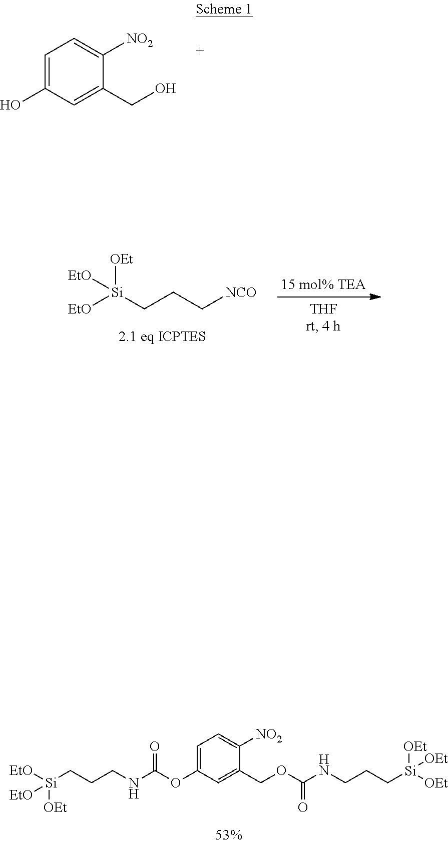

The reaction product could be obtained in a 53% of yield and had been characterized by ¹H-NMR and ¹³C-NMR, FTIR spectroscopy and ESI-mass spectrometry. Furthermore the absorption spectra had been recorded for further light breakability experiments of the linker itself.

### 1.2 Breakability Test on DCNS

The light-induced breakability of the DCNS compound had been performed by irradiating the compound with light produced by a Hg lamp. To this purpose, the compound was dissolved in DMSO-d₆ in a NMR tube. In this way the photo degradation could be followed by recording ¹H-NMR spectra over a certain period of time. Indeed the photogradation reaction could be observed and it is indicated by the signal derived from the aldehyde proton at 10.92 ppm (FIG. 11).

### 1.3 Synthesis of Hybrid MSPs

Firstly, model spherical MSPs were synthesised. The model particles were synthesized according to a modified Stöber synthesis, shown in Scheme 2

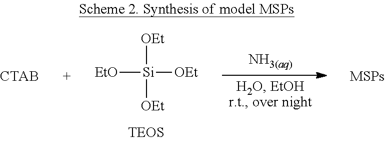 [SMILES: CCO[Si](OCC)(OCC)OCC ; "NH3(aq)" ; "H2O, EtOHr.t., over night"]

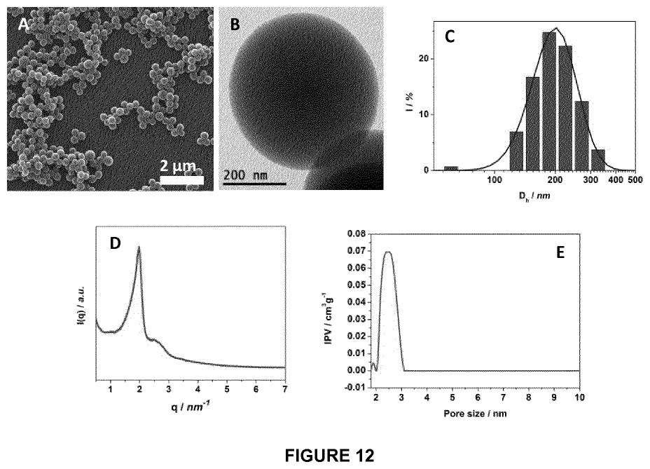

The model particles obtained were spherical characterized by an average diameter of ca 200 nm (SEM, TEM and DLS analysis in FIG. 12). Furthermore these model particles possess a hexagonal mesostructure with an estimated average pore size of 2.5 nm (see SAXS and pore size distribution in FIG. 12)

Once the standard synthesis protocol had been established, hybrid silica particles were synthesised by the co-condensation of DCNS into the silica structure. (see scheme 3)

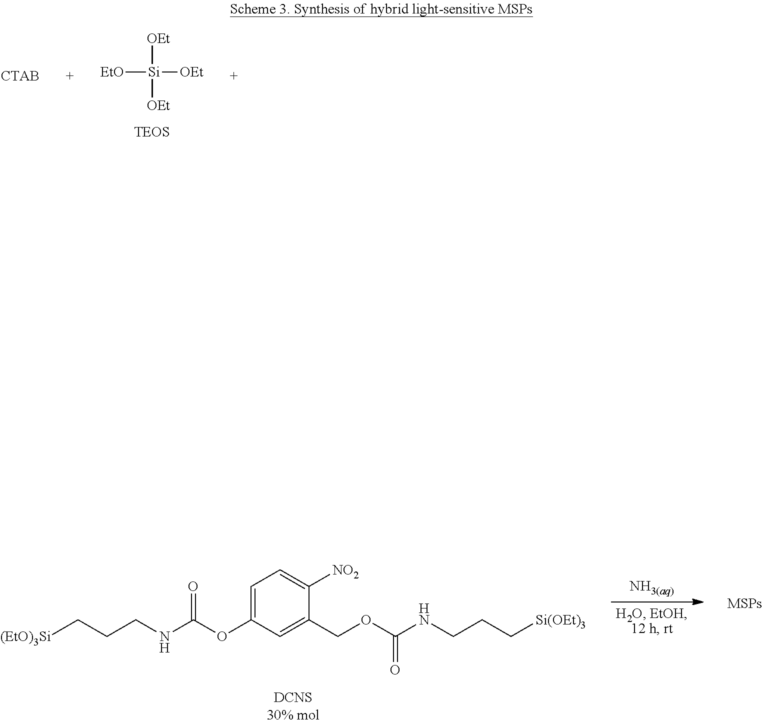

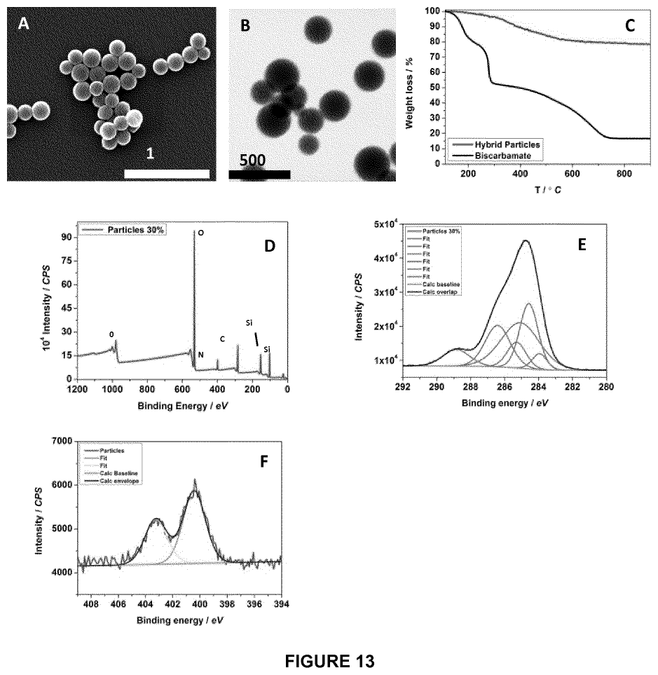

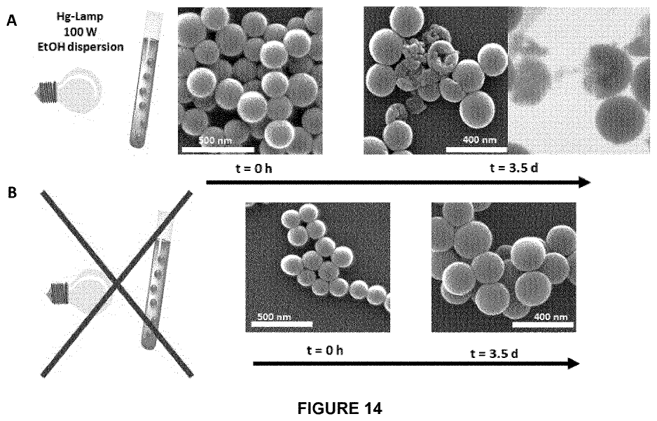

The hybrid silica particles obtained by this synthetic approach were spherical and characterized by satisfactorily monodispersity and diameter of ca. 200 nm and 20 wt. % of organic material as determined by TGA. The incorporation of the DCNS linker was proven by XPS analysis. The deconvolution of high resolution scans of the C(1s) and N(1S) indicated the presence of peaks characteristic for the functional groups present in the linker (FIG. 13). A pH measurement of the reaction mixture before and after the addition of DCNS linker confirmed the hypothesis that the hydrolysis of the carbamate occurs in these conditions. In fact, the pH value changes from 11 to 7. The hydrolysis product could be extracted from the aqueous mixture and the recorded ¹H NMR spectrum showed the presence of a carbamic acid derivative.

The hybrid light-sensitive MSPs may be further functionalized, as described for core/shell nanocapsules above, for covalent incorporation as crosslinkers into hydrogel networks. For example, 40 mg of hybrid light-sensitive MSPs are suspended in 5 mL of ethanol. 44 μL of 3-aminopropyltriethoxysilane (PM=221,37, d=0,946, 0.094 mmol) and 20 μL of triethylamine (TEA) are added to the suspension, that is stirred at R.T. for 18 hours. The resulting NH₂-functionalized hybrid light-sensitive MSPs (NPs, also designated NH2-MSPs herein) are then washed five times with distilled water and dried.

### 1.4 Light Breakability Experiments of Hybrid MSPs

In order to evaluate the light-induced breakability of hybrid light breakable silica particles a suspension of these particles in ethanol was irradiated with a Hg lamp (FIG. 13). As a control experiment, particles were kept in the dark. The SEM and STEM analyses showed that only the particles exposed to light break partially apart, whereas the non-irradiated particles showed no change in morphology. The partial breaking of particles could be explained by the fact, that during the particle formation some of the photolabile linker was hydrolized, while the remaining non-hydrolized linker was incorporated to a certain extent in the hybrid particles.

### 1.5 Synthesis of triethoxy(3-(4-nitro-3-((3-(triethoxysilyl)propoxy)methyl)phenoxy)propyl)silane

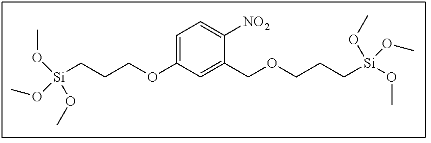

The diether compound can be prepared from 5-hydroxy-2-nitrobenzylalchol through allylation and subsequent hydrosilylation reaction, as depicted in Scheme 4. The synthetic steps are described in detail in Scheme 5.

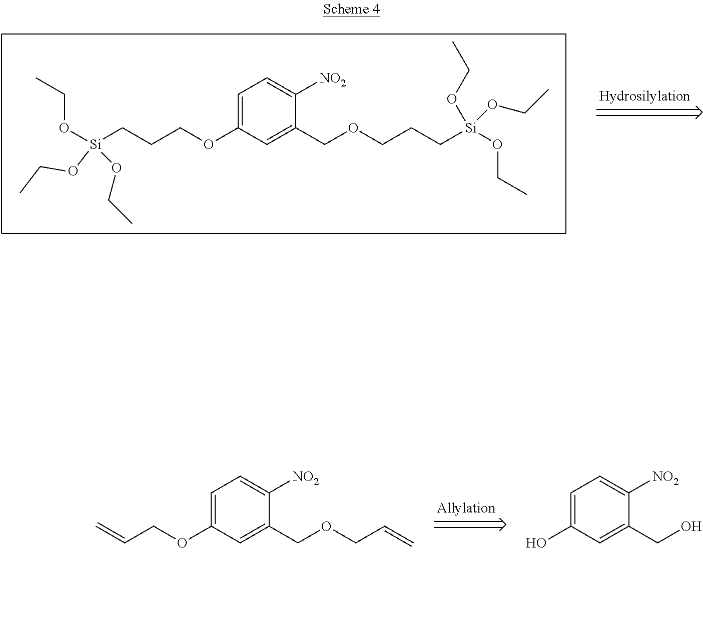

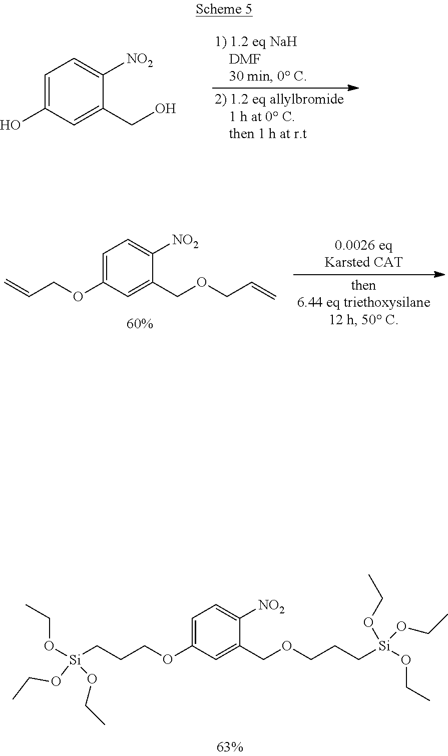

pH-Degradable Hydrogels

1 g of MBA and 250 mg of GABA were weighted in a 50-ml round bottom flask. 0.85 g of diimPEHA were dissolved in 7.5 mL of distilled water and the solution was added to the flask at 45° C. under magnetic stirring until the suspension become clear. The fluid is placed in a glass vial and the temperature is then raised to 60° C.

The experimental protocol described above was repeated with additional different compositions of non degradable (no cleavable crosslinker in the PAAm polymer network) or degradable hydrogels (presence of cleavable crosslinker in the PAAm polymer network). The hydrogel compositions and starting materials are summarized below. Unless otherwise indicated in the table below, all hydrogels formed within 48 hours.

| | | | | | | | | | | | | | | Gel- |
|---|---|---|---|---|---|---|---|---|---|---|---|---|---|---|
| Hy- | | | | | | | | | | | | | | ifica- |
| dro- | Hydro- | | | | | | | | | | | | | tion |
| gel | gel | MONO- | | MONO- | | MONO- | | CROSS- | | | | SOL- | S1 | time |
| num- | Prop- | MER | M1 | MER | M2 | MER | M3 | LINKER | CL1 | OTHER | Other 1 | VENT 1 | amount | (37° |
| ber | erties | 1 (M1) | amount | 2 (M2) | amount | 3 (M3) | amount | 1 (CL1) | amount | 1 | amount | (S1) | (mL) | C.) |
| 5 | Adhe- | MBA | 200,00 | GABA | 34,00 | | | PEHA | 120,00 | | | Water | 1,50 | |
| | sive | (mg) | | (mg) | | | | (μl) | | | | (mL) | | |
| 6 | Degrad- | MBA | 200,00 | DMENA | 38,00 | | | CYST | 130,00 | | | Water | 1,50 | |
| | able | (mg) | | (μl) | | | | (mg) | | | | (mL) | | |
| 7 | Adhe- | MBA | 200,00 | GABA | 50,00 | | | PEHA | 120,00 | PVP | 100,000 | Water | 1,50 | 150 |
| | sive | (mg) | | (mg) | | | | (μl) | | 40,000 | | (mL) | | (RT) |
| | | | | | | | | | | (mg) | | | | |
| 8 | Adhe- | MBA | 200,00 | GABA | 50,00 | | | PEHA | 120,00 | PVP | 25,00 | Water | 1,50 | 150 |
| | sive | (mg) | | (mg) | | | | (μl) | | 40,000 | | (mL) | | (RT) |
| | | | | | | | | | | (mg) | | | | |
| 9 | Adhe- | MBA | 200,00 | GABA | 50,00 | | | PEHA | 120,00 | PVP | 50,00 | Water | 1,50 | 165 |
| | sive | (mg) | | (mg) | | | | (μl) | | 40,000 | | (mL) | | (RT) |
| | | | | | | | | | | (mg) | | | | |
| 10 | Non | MBA | 200,00 | GABA | 50,00 | | | PEHA | 120,00 | | | Water | 1,50 | |
| | Degrad- | (mg) | | (mg) | | | | (μl) | | | | (mL) | | |
| | able | | | | | | | | | | | | | |
| 11 | Non | MBA | 200,00 | GABA | 50,00 | | | PEHA | 70,00 | | | Water | 1,50 | |
| | Degrad- | (mg) | | (mg) | | | | (μl) | | | | (mL) | | |
| | able | | | | | | | | | | | | | |
| 12 | Non | MBA | 200,00 | GABA | 50,00 | | | PEHA | 80,00 | | | Water | 1,50 | |
| | Degrad- | (mg) | | (mg) | | | | (μl) | | | | (mL) | | |
| | able | | | | | | | | | | | | | |
| 13 | Non | MBA | 200,00 | GABA | 50,00 | | | PEHA | 90,00 | | | Water | 1,50 | 110′ |
| | Degrad- | (mg) | | (mg) | | | | (μl) | | | | (mL) | | |
| | able | | | | | | | | | | | | | |
| 14 | Non | MBA | 200,00 | GABA | 50,00 | | | PEHA | 100,00 | | | Water | 1,50 | 88′ |
| | Degrad- | (mg) | | (mg) | | | | (μl) | | | | (mL) | | |
| | able | | | | | | | | | | | | | |
| 15 | Non | MBA | 200,00 | GABA | 65,00 | | | ENA | 35,00 | | | Water | 1,50 | 48 h |
| | Degrad- | (mg) | | (mg) | | | | (μl) | | | | (mL) | | |
| | able | | | | | | | | | | | | | |
| 16 | Non | MBA | 200,00 | GABA | 65,00 | | | ENA | 40,00 | | | Water | 1,50 | 15 h |
| | Degrad- | (mg) | | (mg) | | | | (μl) | | | | (mL) | | |
| | able | | | | | | | | | | | | | |
| 17 | Non | MBA | 200,00 | GABA | 65,00 | | | ENA | 50,900 | | | Water | 1,50 | 8 h 30′ |
| | Degrad- | (mg) | | (mg) | | | | (μl) | | | | (mL) | | |
| | able | | | | | | | | | | | | | |
| 18 | Non | MBA) | 200,00 | GABA | 65,00 | | | ENA | 60,00 | | | Water | 1,50 | 7 h |
| | Degrad- | (mg) | | (mg) | | | | (μl) | | | | (mL) | | |
| | able | | | | | | | | | | | | | |
| 19 | Non | MBA | 200,00 | GABA | 65,00 | | | PEHA | 50,00 | | | Water | 1,50 | |
| | Degrad- | (mg) | | (mg) | | | | (μl) | | | | (mL) | | |
| | able | | | | | | | | | | | | | |
| 20 | Non | MBA | 200,00 | GABA | 65,00 | | | PEHA | 70,00 | | | Water | 1,50 | 200′ |
| | Degrad- | (mg) | | (mg) | | | | (μl) | | | | (mL) | | |
| | able | | | | | | | | | | | | | |
| 21 | Non | MBA | 200,00 | GABA | 65,00 | | | PEHA | 90,00 | | | Water | 1,50 | 4 h |
| | Degrad- | (mg) | | (mg) | | | | (μl) | | | | (mL) | | |
| | able | | | | | | | | | | | | | |
| 22 | Non | MBA | 200,00 | GABA | 65,00 | | | PEHA | 110,00 | | | Water | 1,50 | 4 h |
| | Degrad- | (mg) | | (mg) | | | | (μl) | | | | (mL) | | |
| | able | | | | | | | | | | | | | |
| 23 | Non | MBA | 200,00 | GABA | 65,00 | | | PEHA | 140,00 | | | Water | 1,50 | 2 h 10′ |
| | Degrad- | (mg) | | (mg) | | | | (μl) | | | | (mL) | | |
| | able | | | | | | | | | | | | | |
| 24 | Adhe- | MBA | 200,00 | Dop- | 66,00 | | | PEHA | 89,00 | Ascorbic | 10,00 | Water | 1,50 | |
| | sive | (mg) | | amine | | | | (μl) | | acid | | (mL) | | |
| | | | | (mg) | | | | | | (mg) | | | | |
| 25 | Adhe- | MBA | 200,00 | GABA | 66,00 | | | PEHA | 70,00 | PVP | 50,00 | Water | 1,50 | |
| | sive | (mg) | | (mg) | | | | (μl) | | 40,000 | | (mL) | | |
| | | | | | | | | | | (mg) | | | | |
| 26 | Adhe- | MBA | 200,00 | GABA | 66,00 | | | PEHA | 70,00 | PVP | 100,00 | Water | 1,50 | 150′ |
| | sive | (mg) | | (mg) | | | | (μl) | | 40,000 | | (mL) | | |
| | | | | | | | | | | (mg) | | | | |
| 27 | Adhe- | MBA | 200,00 | GABA | 66,00 | | | PEHA | 70,00 | PVP | 50,00 | Water | 1,50 | |
| | sive | (mg) | | (mg) | | | | (μl) | | 40,000 | | (mL) | | |
| | | | | | | | | | | (mg) | | | | |
| 28 | Adhe- | MBA | 200,00 | GABA | 66,00 | | | PEHA | 70,00 | PVP | 100,00 | Water | 1,50 | 120′ |
| | sive | (mg) | | (mg) | | | | (μl) | | 40,000 | | (mL) | | |
| | | | | | | | | | | (mg) | | | | |
| 29 | Lumin- | MBA | 200,00 | GABA) | 66,00 | | | PEHA | 70,00 | | | MeOH | 1,00 | |
| | escent | (mg) | | (mg) | | | | (μl) | | | | (mL) | | |
| 30 | Lumin- | MBA | 200,00 | GABA | 66,00 | | | PEHA | 70,00 | | | MeOH | 2,00 | |
| | escent | (mg) | | (mg) | | | | (μl) | | | | (mL) | | |
| 31 | Adhe- | MBA | 200,00 | GABA | 67,00 | | | PEHA | 70,00 | | | Water | 1,50 | 200′ |
| | sive | (mg) | | (mg) | | | | (μl) | | | | (mL) | | |
| 32 | Degrad- | MBA | 200,00 | DMENA | 70,00 | | | CYST | 65,00 | | | Water | 1,50 | |
| | able | (mg) | | (μl) | | | | (mg) | | | | (mL) | | |
| 33 | Degrad- | MBA) | 200,00 | DMENA | 86,00 | | | CYST | 32,00 | | | Water | 1,50 | |
| | able | (mg) | | (μl) | | | | (mg) | | | | (mL) | | |

Degradation Kinetic of Stimuli-Responsive Hydrogels For redox-responsive materials, a 1 mm thick hydrogel cylinder is lyophilized and its dry weight is recorded. The hydrogel is then placed in a vial and 5 mL of a 10 μM solution of reduced GSH are added. The swelling of the hydrogel is recorded at the appropriate time-points. The experiment is repeated in triplicated and then with a solution of GSH 10 mM and with a solution of PBS as a reference.

The same procedure was applied for pH-responsive materials, using pH=4 citrate buffer for degradation and PBS as a reference.

Degradation of dPAA hydrogels was examined in the presence of reduced glutathione (GSH), a disulfide reducing agents.

Briefly, the lyophilized hydrogels samples were incubated at 37° C. in 2 mL of a PBS solution with a GSH concentration of 10 μM. dPAA hydrogels were incubated in PBS alone as a control.

The degradation kinetics were then evaluated via swelling ratio (SR) measurements in time.

SR were measured by a gravimetric method. In brief, lyophilized hydrogel samples were immersed in PBS at 37° C. Then, the samples were removed from PBS at set time points (after 1 h, 6 h, 12 h, 24 h, 48 h, 72 h, 144 h), blotted free of surface water using filter paper and their swollen weights were measured on an analytical balance. The SR were then calculated as a ratio of weights of swollen hydrogel (Ws) to dried hydrogel (W), using the following equation:

$$SR = \frac{{W}_{s} - {W}_{d}}{{W}_{d}}$$

Degradation time was defined as the time where there were no longer sufficient crosslinks to maintain the 3D network and the material was completely disintegrated. Experimentally, complete degradation was determined when we could observe a limpid solution, without solid residues.

In Vitro Cell Culturing

Cryopreserved human dermal fibroblast, adult (HDFa) were purchased from Thermo Fisher and the culture was initiated as suggested on the protocol. HDFa were grown in Medium 106 supplemented with Low Serum Growth Supplement (LSGS, Thermo Fisher). Cells were kept in 75 cm² culture flasks (Corning Inc., NY, USA) at 37° C. with a controlled atmosphere of 5% CO₂ and were grown until reaching 80 to 85% of confluence. Then, they were washed twice with PBS and treated with trypsin/EDTA solution to detach them from the flask surface. Cells were split every 2-3 days; the medium was changed every other day.

In Vitro Cell Culturing onto the Nanocomposite Hydrogels

The hydrogel scaffolds equilibrated by adding culture media at 37° C. HDFa were detached from the culture flask by trypsination and approximately 2.5×10⁵ cells were seeded onto the hydrogel scaffolds. Then, the samples were placed in the incubator (37° C., 5% CO₂) for about 30 minutes and fresh media was cautiously added on the top of the hydrogels to supply cells with nutrients. This was done to allow anchorage of the cells onto the scaffolds.

Cell Staining and Viability Studies

Cell viability was assessed using alamarBlue assay. Briefly, the alamarBlue solution was added to the culture medium (1:10 dilution) of unstained cells growing onto hydrogel scaffolds. After 3 h incubation, 200 μL of the media were transferred to a 96-well plate and absorbance signals generated from the dye resazurin (dark blue) being reduced to resorufin (pink) by metabolically active cells were recorded using a VICTOR X5 Multilabel Plate Reader (Perkin Elmer).

Each sample was tested in three replicates and the results were expressed as percentage of reduced alamarBlue.

The viability of cells after complete degradation of the dPAA was measured by with a TC20 (trade mark) Automated Cell Counter (Bio-Rad).

Where required (confocal fluorescence microscopy images), HDFa were stained with Vybrant DiD (Life Technologies, Thermo Fisher Scientific, Waltham, Mass., USA), following the reported protocol, prior to seeding them onto the scaffolds.

Cell-Mediated Degradation of Hydrogel

The hydrogel were freeze-dried and weighed (W). Then 2.5×10⁵ HDFa were seeded onto the samples (see above). The cell-laden samples were collected at pre-determined time points and were freeze-dried to obtain their dry weight after degradation (W).

The cell-mediated degradation of the hydrogels, D, was calculated using the following equation:

$$D ( \% ) = \frac{{W}_{i} - {W}_{f}}{{W}_{i}} \times 100$$

Acellular hydrogels were used as degradation control.

Evaluation of the Gelation and Formation of SFC Ex Vivo

Fresh porcine stomachs were used for the ex vivo tests. The hydrogel solution was injected into the submucosal layers of the pig stomach using a 23-gauge needle. The dose was 2 ml for each sample and the stomach was kept to a temperature of about 37° C. with a lamp to ensure simulation of in vivo conditions. Gelation of the dPAA samples was assessed by cutting open the tissue after the desired time. The experiment was repeated three times.

Creating Submucosal Cushion and Performing ESD in a Living Pig

The pig was fasted for 1 day before operation.

Endoscopy was performed by the surgeon.

A standard endoscope (Karl Storz, Tuttlingen, Germany) was used in the pig under general anesthesia. Both the dPAA solution and the NS used as control contained a small amount of Methylene Blue as a color agent in order to facilitate visualization of the SFC.

After setting appropriate lesion sizes of approx. 3 cm in diameter in the porcine stomach, 810 ml of dPAA solution and NS were injected in the stomach submucosa through the endoscope accessory channel using a 23-gauge injection needle.

The mucosal elevation due to the injected dPAA at the target site was observed endoscopically before starting the ESD. It was compared under direct view with the elevation caused by NS during the procedure.

After injection, the ESD was performed and a circumferential mucosal incision was accomplished using a Needle knife (Olympus, Tokyo, Japan)

Injection of dPAA and ESD were repeated three times.

The animal was euthanized after completion of experiments; the whole procedure was followed and recorded using a Silver Scope™ Video Gastroscope (Karl Storz, Tuttlingem, Germany).

The main outcome measures were (1) the rapid gelation of dPAA when injected into the submucosa and (2) the long-lasting SFC formed; (3) the feasibility of the dissection procedure during ESD; (3) the adhesion of dPAA to the muscolaris layer and thus the increase of protection during the procedure and after it.

### Study Design

A degradable nanocomposite hydrogel, also called dPAA below, was synthetized and characterized. It is composed of a polyamidoamines-based network with embedded breakable silica hollow nanocapsules, BNCs.

Both, the BNCs and the polymeric backbone of the scaffold contain disulfide linkers that could be cleaved in presence of glutathione (GSH). The nanocomposite could be completely degraded even at a very low concentration of GSH (i.e. 10 μM), which was chosen to mimic the extra-cellular environment. Degradation and release kinetics of model protein cytochrome, loaded into the particles, were evaluated.

Next, the cell-mediated degradation of dPAA in the presence of adult Human Dermal Fibroblasts (HDFa) proliferating onto the scaffolds was tested. The assay demonstrated the achievement of cell-controlled degradation of the material; complete dissolution of the scaffold was observed after 96 hours when 2.5×10⁵ cells were seeded onto the nanocomposite.

Then, the injection of the hydrogel solution (i.e. before complete gelation) was attained through a 23-gauge needle and the formation of the hydrogel was evaluated ex vivo. This allowed us to observe a fast gelation (<10 minutes), probably due to the increase of temperature and the interactions between the dPAA and the collagen present in the submucosal layer, where the injection was performed. Moreover, the dPAA was able to provide a stable and long-lasting mucosal elevation when tried as SFC.

Finally, the dPAA was tested as SFC for ESD procedures in vivo, on a porcine stomach.

The formation of the hydrogel and SFC in vivo was observed after 3 minutes and allowed the surgeon to perform the ESD procedure with a single injection. The adherence of part of the dPAA to the muscularis layer not only protected it during the procedure but also potentially offers several advantages in the phase following the surgery. The cell-mediated degradation of the nanocomposite indeed has shown to lead to the release of the active component loaded into the particles. This behavior could be exploited to release antibiotics or active factors to assist the healing of the wounded tissue and finally to achieve a complete clearance of the hydrogel form the body.

### Synthesis of the Materials

### Synthesis of Breakable Nanocapsules

The nanocapsules used are those disclosed in E. A. Prasetyanto, A. Bertucci, D. Septiadi, R. Corradini, P. Castro-Hartmann, L. De Cola, Angew. Chem. Int. Ed. 2016, 55, 3323. [21]. This platform is composed of a silica shell able to encapsulate functional proteins in their active folding and it is engineered to degrade upon contact with a reducing agent, such as GSH present in the biological environment with a complete release of the loading.

An hydrogel comprising these BNCs to construct hydrogels comprising nanoparticles or nanocapsule able to release active molecules during the degradation of the material.

Cytochrome C (Cyt-C) was chosen as model cargo, since its strong absorption in the visible region allowed us to investigate the release kinetics during the hydrogel degradation.

The synthesis of the BNCs, in order to prevent denaturation of the loaded active molecule, was performed following the reported reverse nano-emulsion procedure. In particular, the silica precursor, tetraethyl orthosilicate (TEOS) was added to bis[3-(triethoxysilyl)propyl]disulfide in a ratio 7:3 TEOS:bispropyldisulfide derivative, in order to introduce the redox-sensitive moiety. Well-defined and monodispersed spherical nanocapsules with a diameter of around 60±10 nm were obtained.

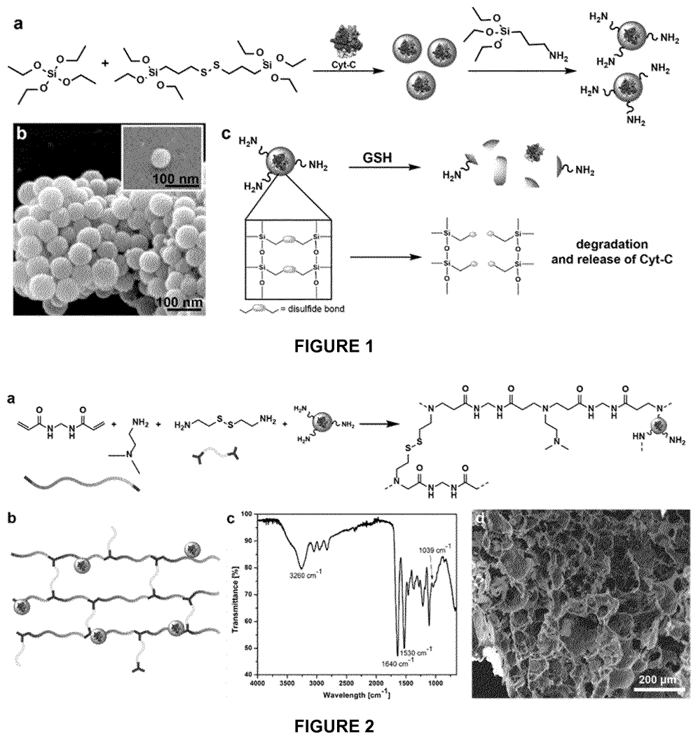

Then, the obtained pristine BNCs were functionalized with 3-aminopropyltriethoxysilane, to be able to covalently link the BNCs to the polymeric hydrogel network. A scheme of the synthesis and functionalization, as well as the SEM of the pristine and functionalized BNCs and of the degradation via GSH is displayed in FIG. 1.

The surface functionalization was confirmed by the shift from negative to positive values of the ζ-potential, from −10.5 mV of the pristine nanocapsules to ±2.2 mV.

Then, the functionalized BNCs (1 mg/ml) were used to synthetize the dPAA nanocomposite hydrogel through surface-grafting of the aminated BNCs to the polyamidoamine backbone of the scaffold.

Synthesis of the Nanocomposite Hydrogel, dPAA

Advantageously the inventors designed the network of the dPAA to achieve a degradation that could be triggered by cells proliferating onto the material, without the need of any additional stimulus. Thus, a crosslinker, for example a disulfide, was incorporated in the polymeric network of the hydrogel, i.e. cystamine. Disulfide bonds are susceptible of thiol exchange in the presence of reducing agents, such as glutathione (GSH), which is a cell metabolite.

The inventors demonstrated that the reducing microenvironment given by the presence of GSH in the extra-cellular environment could trigger the cleavage of the disulfide bonds, therefore providing the dissolution of the scaffold.

Disulfide-modified polyamidoamines-based hydrogels containing BNCs were synthesized following the previously reported method with some modifications.^{[17]} In particular, amino groups on BNCs were reacted with the unsaturated moiety of methylenebisacrylamide (MBA) through a one-pot Michael poly-addition in water, at room temperature (FIG. 2a)

Briefly, a mixture of MBA, and N,N-dimethylethylenediamine, DMEN, was stirred in a BNCs water dispersion (1 mg/ml) at room temperature in presence of cystamine. A transparent liquid was obtained after 30 minutes, and then was left in static conditions to complete the gelation process in 2 days. FIGS. 2a,b show a schematic representation of the synthesis and structure of the nanocomposite network.

The synthesis afforded transparent hydrogels formed in water at room temperature using a catalyst-free Michael-type addition. Gelation was confirmed by the absence of gravitational flow when the test tubes containing the hydrogels were inverted, through the so called “inverted test tube method”.

The formation of a crosslinked network was further confirmed by Fourier transformed infrared spectroscopy (FTIR), showed in FIG. 2c. In particular, peaks at 1640 and 1530 cm⁻¹ are typical for the absorption of the amides carbonyl (st and v of C═O), while the 3260 cm-¹ band is attributed to the N—H stretching (st NH₂). Furthermore, the absorption band at 1039 cm⁻¹, which was ascribed to the vibration of C—S—S—C bond, confirmed of the successful incorporation of the disulfide cross-linker in the polymeric network. Since the samples were lyophilized to allow a correct peak determination, the presence of adsorbed water cannot be seen from FTIR spectrum, apart from a weak residual shoulder at 3430 cm⁻¹, which is related to the OH angular deformation of water.

The morphological analysis of the obtained hydrogel scaffolds was assessed via scanning electron microscopy (SEM) of the lyophilized scaffolds. SEM showed a highly porous structure, with pores diameter in the range of 40 to 100 m, as can be seen in FIG. 2d.

### Degradation and Release Kinetics

### Degradation in Presence of GSH

For some medical application, hydrogels should maintain the required mucosal elevation for a determined time (i.e. 30 min to 1 hour), and then degrade into fragments, in order to have a complete clearance from the body.

The nanocomposite presented in this work was degradable upon exposure to GSH, via the incorporation of cystamine cross-links throughout the polymeric network and in the particle (BNCs) framework. The potential degradation mechanism of the network is shown in FIG. 7.

The degradation kinetics of the dPAA was examined by measuring swelling ratio variations as function of time in the presence of a low concentrated GSH solution (i.e. 10 μM GSH solution in PBS), mimicking the extracellular environment. Hydrogels, incubated in PBS in the absence of GSH were used as control.

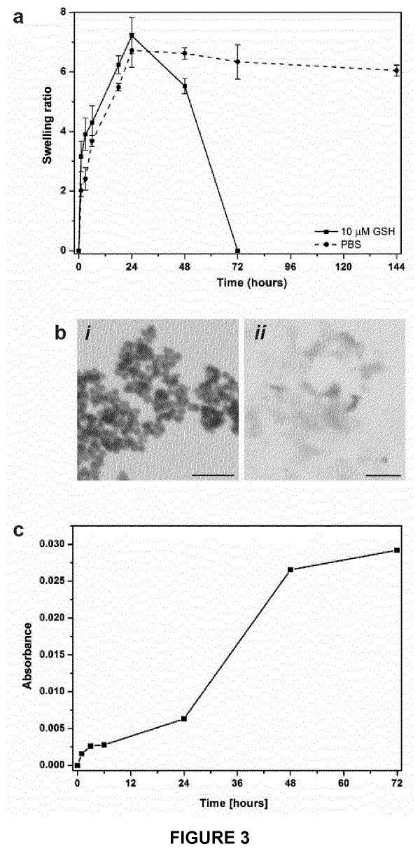

The swelling ratio curve of dPAA showed two different phases: an initial phase where clear increase in swelling was observed, followed by a rapid downward phase (FIG. 3a).

The imbibing of the solvent into the hydrogel caused the initial increasing phase. This was then quickly outweighed by the cleavage of the disulfide bonds, leading to the complete degradation of the hydrogel.

A clear point of reverse gelation, defined as the point where there are less than 2 crosslinks per polymer chain and the branched polymer chains dissolve,^{[22]} was identified after 24 hours.

As disulfide bonds were cleaved, mass loss increases with time until there is no longer a sufficient number of crosslinks to maintain the 3D network. Finally, the equilibrium swelling value was found equal to zero after 3 days, due to complete disintegration of the hydrogel network.

The dPAA equilibrated in PBS showed instead a first phase of swelling followed by a plateau that was reached after 24 hours, demonstrating that the nanocomposite is stable in absence of reducing agent. The swelling was followed for 6 days.

The fast degradation rate observed, even with a low concentration of GSH, could be ascribed to the chemical environment of the hydrogel network surrounding the disulfide moieties, as shown in other studies.^{[20b]} It has been reported that the chemical environment of the disulfide units plays a key role in determining their degradation kinetics.^{[23]} Thus, the presence of electronegative groups in the adjacency of the disulfide bonds in the dPAA make them more susceptible to cleavage and hence, lead to a fast degradation.

### Tunability of the Degradation Kinetic

To evaluate the effect of the disulfide crosslinker density on the degradation kinetics of the hydrogel nanocomposite, other two samples were synthetized. These scaffolds had the same composition of the dPAA, except for the amount of cystamine. In particular, they contained a lower and a higher amount of cystamine, 10-wt % and 40-wt %, compared to the dPAA hydrogel, which had 20 wt %.

In this way, a range of degradation profiles and times that could be achieved in response to GSH reducing microenvironments and that could be controlled by adjusting the molar ratio of the disulfide bond were established.

The degradation kinetics were evaluated by immersing the samples in the reducing solution ([GSH]=10 μM) and by measuring the swelling ratio after precise time intervals, as already described above.

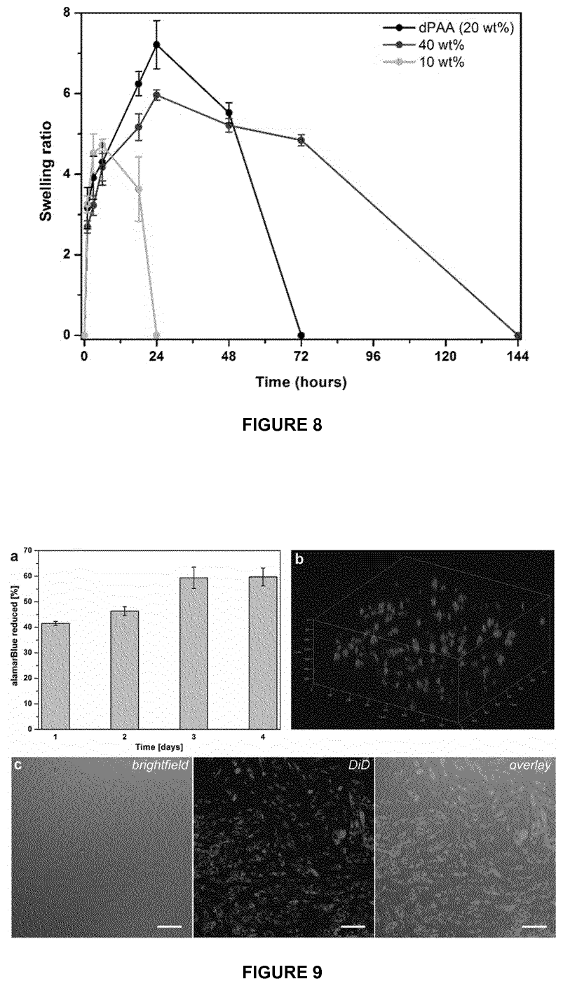

The degradation profiles of the three samples are reported in FIG. 8.

The degradation time was found to be proportional to the amount of disulfide crosslinker; in particular a decrease was observed with the scaffold containing 10 wt % of cystamine, which completely after 24 hours. Instead the sample crosslinked with an higher amount of cystamine (40 wt %) displayed a longer degradation profile, terminating after 6 days with the complete disintegration of the network.

The possibility of tuning the degradation kinetic of the nanocomposite scaffold developed by changing the ratio of the disulfide linker, demonstrate its potential for applications where a precise control of the breakability over time is required.

Release of Model Protein Cyt-C

As mentioned in the previous sections, the degradable hydrogel was decorated with breakable nanocapsules able to degrade with the same mechanism of the hydrogel, through the reduction of the disulfide bonds, and able to release their content.

The fragmentation of the BNCs in presence of the reducing GSH after 72 hours, was further confirmed by scanning transmission electron microscope, as shown in FIG. 3b.

Cytochrome-C, Cyt-C, was used as a model protein to study the release kinetics thanks to its strong absorption in the visible region, due to the presence of the eme group.

The BNCs were thus embedded into the dPAA and the Cyt-C released from the nanocomposite was investigated, by incubating the scaffold in the 10 μM solution of GSH in PBS.

The cumulative release of Cyt-C from the dPAA is reported in FIG. 3c and shows a slow release in the first 6 hours, followed by an increase of detected Cyt-C. The release was observed by measuring the absorbance of the solution at 410 nm. The release of Cyt-C from the scaffold is highly augmented by the degradation of the hydrogel structure, which can be seen from the shape of the curve between 24 and 72 hours, showing a steep growth in absorption. The overall amount of protein release from the scaffold was estimated to be 67% of the initial loading.

Overall, as demonstrate the incorporation of cleavable groups, both in the embedded NPs and into the hydrogel network, which can degrade in response to endogenous stimuli, is an attractive strategy for in vivo procedures, such as ESD. The system is completely cleared from the body and release molecules of choice during the degradation process, such as a drug to assist the healing of the wound or in situ chemotherapy. The versatility of the synthesis allows the tailor-made nanocomposite hydrogel preparation in response to the needs of individual patients.

### In Vitro and Ex-Vivo Analyses

### Cell-Mediated Degradation

Since the degradation of hydrogel and the release of the model protein was achieved at low GSH concentration, such as the extra-cellular one, the degradation of the scaffold in the presence of cells was tested. In particular, Human Dermal Fibroblast (HDFa) were chosen for this study because fibroblasts residing within the extracellular matrix in the body are critical for matrix synthesis and repair. Upon injury or wound formation, these cells migrate to the wound site to repair the damaged tissue.^{[24]} Thus, to simulate the cell-mediated degrading conditions in vivo, we selected HDFa.

The dPAA hydrogels for this test were synthetized in a 8 mm diameter and −1 mm height disc shape and. 2.5×10⁵ HDFa were seeded onto the hydrogels and cultured in the corresponding growth medium; acellular hydrogels incubated in growth medium were used as control.

AlamarBlue assay indicated that the majority of the encapsulated cells were viable and proliferating onto the scaffold (FIG. 9a) up to 4 days. This is consistent to what has been observed for similar polyamidoamines-based scaffolds and it confirmed that hydrogel containing disulfide moieties supported cell encapsulation and viability. FIG. 9b displays an image of the 3D proliferation of the cells stained in red (Vibrant DiD stain) for better visualization, indicating that they permeate in the depth of the scaffold. A 3-channel visualization of the surface of the dPAA is reported in FIG. 9c and is indicative of the growth of the cells onto the scaffold.

The hydrogel underwent degradation responding to cell-secreted GSH, in the absence of any external stimulus. In addition, many cell surface molecules contain thiol groups and thus could contribute to the cleavage of the disulfide bonds of the network.

The scaffold resulted largely reduced in size and weight after 72 hours and the complete degradation was achieved after 96 hours.

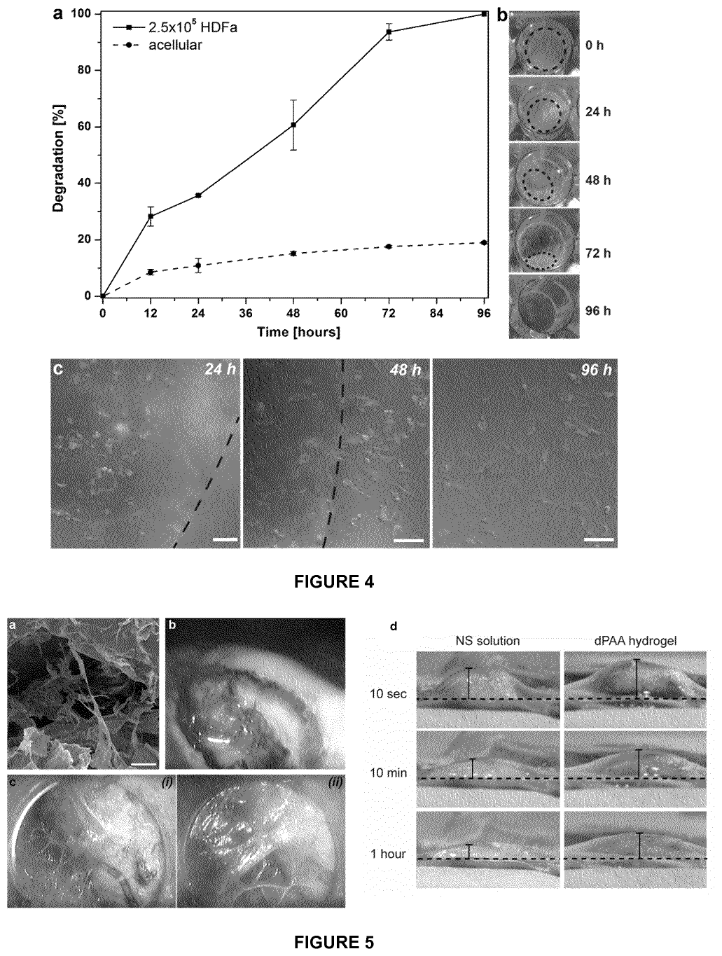

FIG. 4a,b), demonstrating a good agreement with the GSH degradation tests.

It was observed that the degradation process resulted into a gradual movement of the cells from the nanocomposite to the bottom of the well containing the scaffold (FIG. 4c). The viability of HDFa measured after the degradation of the scaffold showed that 87% of the cells were viable, thus confirming that the degradation products were non-toxic. Previous studies on linear polyamidoamines systems have shown that the degradation products were completely non-cytotoxic, since the degradation produced oligomers.^{[25]}

The acellular hydrogels used as control displayed minimal degradation during the course of the studies (FIG. 4a, dashed line). This small degradation (17%) was probably due to the presence of fetal bovine serum in the culture medium, which contains various proteins and amino acids with thiol groups.

The obtained results clearly demonstrate that the dPAA hydrogels were prone to HDFa-mediated degradation through thiol reductive exchange, therefore showing potential for in vivo applications requiring degradation of the scaffold.

### Hydrogel: Injectability and Formation of SFC

The hydrogel according to the invention is a material that could be delivered in vivo via injection, and then rapidly gel inside the body.

Polyamidoamine-based hydrogels have the great advantage of allowing network formation under physiological conditions.

Thus, taking advantage of the catalyst-free water-based reaction through which hydrogels could be obtained, and having observed a time window of several hours between the beginning of the polymerization and the complete gelation, the obtained scaffold for the injection procedure was tested.

When the polyaddition reaction between MBA and DMEN was carried out using the biodegradable cystamine crosslinker, we observed the formation of a clear solution after 30 minutes, indicating that the reagents were completely dissolved and have started the polyaddition reaction.

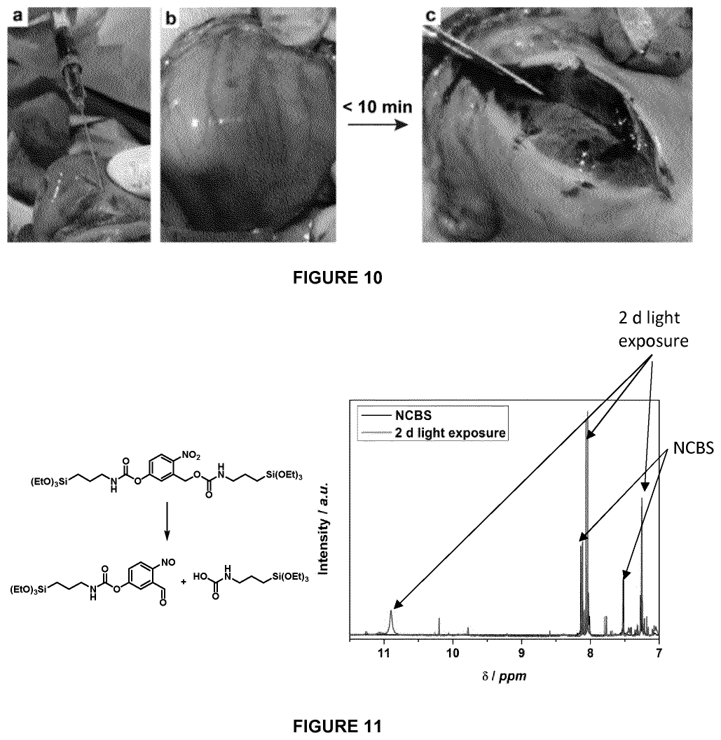

The ability of the hydrogel solution to flow through a disposable 23-gauge catheter injection needle was then examined. The dPAA solution was able to flow under hand pressure and the maximum needle injection pressure was found to be comparable to saline solution (FIG. 10a). Then, we observed that this viscous hydrogel solution reacted at room temperature in static conditions to form a self-standing hydrogel after 48 h.

Better results were obtained by raising the temperature to 37° C., when we observed the formation of a self-standing hydrogel in 18 hours.

However, knowing that intestinal submucosa is rich in type I collagen and that this presents amino and hydroxyl groups as side groups, the possible formation of hydrogen bonds that could lead to a faster gelation kinetic in vivo was tested.

Thus the dPAA solution was injected ex vivo in the submucosa of a porcine stomach. The injection was performed on the tissue at 37° C. and the gelation, with formation of a SFC, was immediately observed (FIG. 10b). There was no leaching of the solution away from the injected site, and the tissue was cut opened, revealing the formation of the dPAA after 8-10 min post injection ex vivo (FIG. 10c).

The nanocomposite hydrogel was found completely adhered to the submucosal layer and it had to be removed with scissors and tweezers. Moreover, it was confirmed that there had not been diffusion of the solution into the surrounding tissues.

The investigation of injectability and gelation time was then performed in vivo. The hydrogel (dPAA) showed a gelation time of approximately 3 minutes when injected in the submucosal layer in a living pig.

As demonstrated, unexpectedly and advantageously, physiological conditions of temperature (37° C.) and pH (7.4), contribute to the faster gelation than what observed in vitro, as already reported for similar systems.^{[28]}

The intermolecular interaction between the hydroxyl and amino groups of the collagen side chains with amide groups of dPAA lead to the formation of hydrogen bonds that further crosslink the polymer network.

Moreover, the formation of mechanical entanglements between the collagen fibers and the dPAA backbone advantageously may also contribute to the formation of an interpenetrated hydrogel network, favoring the faster formation of a stable and elastic hydrogel in situ. This behavior was observed via SEM of the explanted tissues, which showed interactions between the hydrogel scaffold and collagen fibers (FIG. 5a).

In vivo images of the dPAA formed in situ (FIG. 5b) and stained with Methylene Blue also displayed fibrous formation within the hydrogel matrix (FIG. 5c).

Thus, the formation and lasting of a SFC ex vivo by the hydrogel dPAA and a NS was examined. In particular, fresh resected porcine stomachs were used, and 2 ml of NS or DPAA solution were injected. Protrusions appeared at the injection site and the height changes in submucosal elevation were recorded after 10 seconds, 10 minutes and 1 hour, to cover the whole time of the ESD procedure, which is approximately 40 minutes (FIG. 5d).

Although both the examined solution and the hydrogel led to the mucosal elevation right after the injection, the hydrogel according to the invention comprising nanocomposite displayed higher mucosal lifting, 8.3 mm vs 6.7 mm, for the dPAA and the NS respectively, with the same amount of solution injected. This showed that already part of the NS solution was absorbed by the surrounding tissues after 10 seconds.

After the injection of the hydrogel solution, the formation of a solid SFC was detected, which showed only a slight change in size over 1 hour, i.e. from 8.3 mm to 5.8 mm. No significant change in shape or consistency of mucosal lifting was observed. This behavior was due to the formation of the dPAA hydrogel under the submucosa.

In contrast, the elevation created with NS gradually collapsed, showing a reduction of 37% in size after 10 minutes and of more than half after 1 hour (i.e. height from 6.7 mm to 2.9 mm).

In short, this example demonstrates the higher performance of the hydrogel of the invention nanocomposite in the formation of a higher and longer lasting mucosal elevation.

In Vivo ESD Procedure (Endoscopic Submucosal Dissection) A feasibility study to evaluate the in vivo efficacy of hydrogel according to the invention (dPAA) in a living pig was performed. We first set appropriate lesion sizes of approx. 3 cm in diameter in the porcine stomach and then 8-10 ml of the hydrogel solution were injected in the submucosa.

The ESD procedure was performed in triplicate in different areas of the same porcine stomach; NS solution was used as control.

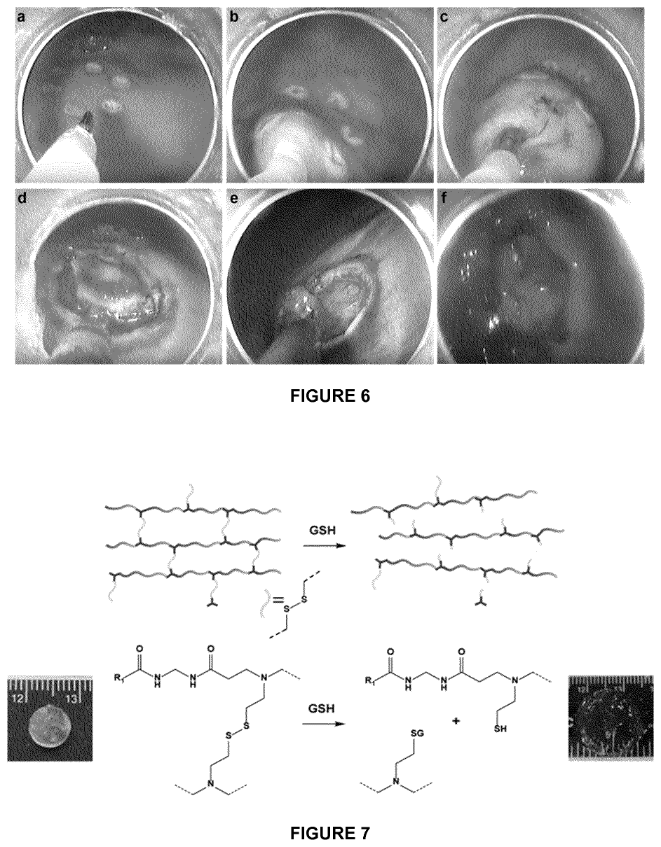

In FIG. 6a is reported the endoscopic view of the stomach at time 0 before the injection of the pre-hydrogel solution. It shows the set of an appropriate lesion of approx. 3 cm in diameter.

In all the cases the injected hydrogel solutions according to the invention in the submucosa led to the gelation of the material in 3 minutes, which thus formed a clear and stable mucosal elevation (FIG. 6b,c).

A comparison with the normal saline solution generally used showed initially no significant difference compared to the SFC formed by hydrogel of the invention (dPAA). However, the elevation of the SFC formed by NS had obviously reduced after 15 min, due to quick diffusion of the NS at the target site and absorption of the liquid by the tissue, thus it was necessary to repeated the injections to keep the lifted submucosa and be able to finish the surgery.

In contrast, the mucosal lifting obtained with dPAA allowed the surgeon to perform the entire ESD procedure (40 min) without requiring a second injection, therefore significantly simplifying the procedure and avoiding large injection of liquids.

The presence of the hydrogel allowed the use of the common electrocautery settings and the long-lasting conservation of the mucosal elevation created with the dPAA enabled the surgeon to smoothly accomplish the circumferential submucosal resection (FIG. 6d). This demonstrate that no significant complications or perforation occurred during the procedure due to the reliable and long-lasting mucosal lifting achieved with the dPAA.

Then, the lesion was dissected en bloc without any sign of mucosal or muscularis damage, confirming that the dPAA hydrogel formed in situ was able to “dissect” the submucosal layer (FIG. 6e). Intact mucosal specimens were conveniently achieved via the performed ESD procedures. This is essential, as a definite resection provides accurate histological assessment and thus, can reduce the risk of neoplastic recurrence.

The same procedure was performed in the colon and in the esophagus with excellent results too.

Part of the hydrogel stayed under the resected mucosa was observed. In this way a protecting layer of hydrogel was obtained onto the newly formed cavity (FIG. 6f).

The possibility of releasing an active component from the dPAA during its degradation is highly beneficial at the end of such delicate procedure. Biomolecules, such as adrenaline, proton-pump inhibitors or antibiotics could potentially be efficiently delivered to assist the cauterization of the resected tissue or the prevention of inflammations.

Moreover, the design of the nanocomposite hydrogel enhances the versatility of the system, enabling the selection of different possible releasing factors, personalized in relation to the patient's requirements.

The reported non-survival animal study was conducted to examine the formation of SFC from the dPAA in vivo and the feasibility of ESD procedure with the novel material.

### CONCLUSION

A hydrogel of the invention, in particular a degradable nanocomposite hydrogel was successfully developed by embedding breakable nanocapsules into a disulfide-containing polyamidoamines-based hydrogel.

In addition, a degradable nanocomposite hydrogel was successfully developed by embedding breakable nanocapsules into a disulfide-containing polyamidoamines-based hydrogel.

The example demonstrate that disulfide bonds of the embedded BNCs and of the network can be completely cleaved in 3 days when the hydrogel is incubated in a GSH solution mimicking the extra-cellular concentration (10 μM).

Most importantly, an example of hydrogel according to the invention sustained the proliferation of HDFa and underwent complete degradation in response to cell-secreted molecules from HDFa seeded onto the scaffold without any external stimulus.

The degradation of the nanocomposite allowed the release of a model protein encapsulated into the BNCs.

Advantageously, the obtained hydrogel according to the invention can be delivered to the desired tissue, for example by facile injection through a 23-gauge needle. Its applicability in-vivo models was proved: the aqueous hydrogel solution was injected in the submucosa of a porcine stomach in vivo. It formed an elastic hydrogel in 3 minutes most likely due to temperature increase and interaction with collagen fibers present in the submucosal layer of the mammals.

Such an important result demonstrates that the hydrogel according to the invention is a novel injection agent, for example subcutaneous, for example for use in ESD.

The example also demonstrate that the hydrogel formed a reliable SFC in vivo, enabling a long-lasting mucosal elevation, which was superior to commonly used NS. This facilitated en bloc resection of the lesion, which was successfully accomplished with just a single injection.

No perforation, major bleeding or tissue damage were observed during ESD. Moreover, part of the in situ formed hydrogel adhered tightly to the muscolaris, under the resected mucosa, allowing protection of the membrane during the procedure and after it.

The results demonstrate a slow degradation kinetics and parallel release of the chosen active molecule in vitro throughout a period of 72 hours, triggered by the proliferation of cells into the scaffold.

Thus, a similar behavior is obtained in vivo, allowing the release of antibiotics, drugs or proteins such as that could assist the healing of the resected tissue during the degradation of the material, and finally the complete clearance of the scaffold.

### LIST OF REFERENCES

[1] J. Ferlay, H. R. Shin, F. Bray, D. Forman, C. Mathers, D. M. Parkin, Int. J. Cancer 2010, 127, 2893.
[2] a) H. H. Hartgrink, E. P. M. Jansen, N. C. T. van Grieken, C. J. H. van de Velde, The Lancet, 374, 477; b) J. A. Ajani, D. J. Bentrem, S. Besh, T. A. D'Amico, P. Das, C. Denlinger, M. G. Fakih, C. S. Fuchs, H. Gerdes, R. E. Glasgow, J. A. Hayman, W. L. Hofstetter, D. H. Ilson, R. N. Keswani, L. R. Kleinberg, W. M. Kom, A. C. Lockhart, K. Meredith, M. F. Mulcahy, M. B. Orringer, J. A. Posey, A. R. Sasson, W. J. Scott, V. E. Strong, T. K. Varghese, G. Warren, M. K. Washington, C. Willett, C. D. Wright, N. R. McMillian, H. Sundar, J. Natl. Compr. Canc. Netw. 2013, 11, 531; c) A. G. Zauber, S. J. Winawer, M. J. O'Brien, I. Lansdorp-Vogelaar, M. van Ballegooijen, B. F. Hankey, W. Shi, J. H. Bond, M. Schapiro, J. F. Panish, E. T. Stewart, J. D. Waye New Engl. J. Med. 2012, 366, 687.
[3] S. Coda, S.-Y. Lee, T. Gotoda, Gut and Liver 2007, 1, 12.
[4] a) S. Oka, S. Tanaka, I. Kaneko, R. Mouri, M. Hirata, T. Kawamura, M. Yoshihara, K. Chayama, Gastrointest. Endosc. 2006, 64, 877; b) T. Gotoda, H. Yamamoto, M. R. Soetikno, J. Gastroenterol. 2006, 41, 929.
[5] a) M. Fujishiro, N. Yahagi, K. Kashimura, T. Matsuura, M. Nakamura, N. Kakushima, S. Kodashima, S. Ono, K. Kobayashi, T. Hashimoto, N. Yamamichi, A. Tateishi, Y. Shimizu, M. Oka, M. Ichinose, M. Omata, Gastrointest. Endosc. 2005, 62, 933; b) K. B. Cho, W. J. Jeon, J. J. Kim, World Journal of Gastroenterology: WJG 2011, 17, 2611.
[6] a) L. Yu, W. Xu, W. Shen, L. Cao, Y. Liu, Z. Li, J. Ding, Acta Biomater. 2014, 10, 1251; b) M. Fujishiro, N. Yahagi, K. Kashimura, Y. Mizushima, M. Oka, S. Enomoto, N. Kakushima, K. Kobayashi, T. Hashimoto, M. Iguchi, Y. Shimizu, M. Ichinose, M. Omata, Endoscopy 2004, 36, 579.
[7] K. J. Kang, B.-H. Min, J. H. Lee, E. R. Kim, C. O. Sung, J. Y. Cho, S. W. Seo, J. J. Kim, Dig. Dis. Sci. 2013, 58, 1491.
[8] R. T. Tran, M. Palmer, S.-J. Tang, T. L. Abell, J. Yang, Gastrointest. Endosc. 2012, 75, 1092.
[9] A. O. Ferreira, J. Moleiro, J. Torres, M. Dinis-Ribeiro, Endoscopy International Open 2016, 4, E1.
[10] a) T. Uraoka, T. Fujii, Y. Saito, T. Sumiyoshi, F. Emura, P. Bhandari, T. Matsuda, K.-I. Fu, D. Saito, Gastrointest. Endosc. 2005, 61, 736; b) M. Fujishiro, N. Kakushima, S. Kodashima, K. Kashimura, T. Matsuura, Y. Muraki, A. Tateishi, M. Omata, Digestive Endoscopy 2007, 19, 26.
[11]A. B. Feitoza, C. J. Gostout, L. J. Burgart, A. Burkert, L. J. Herman, E. Rajan, Gastrointest. Endosc., 57, 41.
[12]N. Yoshida, Y. Naito, M. Kugai, K. Inoue, K. Uchiyama, T. Takagi, T. Ishikawa, O. Handa, H. Konishi, N. Wakabayashi, N. Yagi, S. Kokura, Y. Morimoto, K. Kanemasa, A. Yanagisawa, T. Yoshikawa, J. Gastroenterol. Hepatol. 2011, 26, 286.
[13] Y. Matsui, M. Inomata, K. Izumi, K. Sonoda, N. Shiraishi, S. Kitano, Gastrointest. Endosc. 2004, 60, 539.
[14] T. Hayashi, T. Matsuyama, K. Hanada, K. Nakanishi, M. Uenoyama, M. Fujita, M. Ishihara, M. Kikuchi, T. Ikeda, H. Tajiri, Journal of Biomedical Materials Research Part B: Applied Biomaterials 2004, 71B, 367.
[15] I. Kumano, M. Ishihara, S. Nakamura, S. Kishimoto, M. Fujita, H. Hattori, T. Horio, Y. Tanaka, K. Hase, T. Maehara, Gastrointest. Endosc. 2012, 75, 841.
[16] J. L. Ifkovits, J. A. Burdick, Tissue Eng. 2007, 13, 2369.
[17] F. Fiorini, E. A. Prasetyanto, F. Taraballi, L. Pandolfi, F. Monroy, I. López-Montero, E. Tasciotti, L. De Cola, Small, accepted.
[18] L. Maggini, I. Cabrera, A. Ruiz-Carretero, E. A. Prasetyanto, E. Robinet, L. De Cola, Nanoscale 2016, 8, 7240.
[19] S. Mura, J. Nicolas, P. Couvreur, Nat Mater 2013, 12, 991.
[20] a) F. Yang, J. Wang, G. Peng, S. Fu, S. Zhang, C. Liu, J. Mater. Sci. Mater. Med. 2012, 23, 697; b) M. Kar, Y.-R. Vernon Shih, D. O. Velez, P. Cabrales, S. Varghese, Biomaterials 2016, 77, 186.
[21] E. A. Prasetyanto, A. Bertucci, D. Septiadi, R. Corradini, P. Castro-Hartmann, L. De Cola, Angew. Chem. Int. Ed. 2016, 55, 3323.
[22] a) G. D. Nicodemus, S. J. Bryant, Tissue Engineering. Part B, Reviews 2008, 14, 149; b) H. Henning Winter, in Encyclopedia of Polymer Science and Technology, John Wiley & Sons, Inc., 2002.
[23] A. J. Parker, N. Kharasch, Chem. Rev. 1959, 59, 583.
[24] M. E. Smithmyer, L. A. Sawicki, A. M. Kloxin, Biomaterials Science 2014, 2, 634.
[25] P. Ferruti, S. Bianchi, E. Ranucci, F. Chiellini, V. Caruso, Macromol. Biosci. 2005, 613.
[26] M. P. Lutolf, J. A. Hubbell, Biomacromolecules 2003, 4, 713.
[27] C. D. Pritchard, T. M. O'Shea, D. J. Siegwart, E. Calo, D. G. Anderson, F. M. Reynolds, J. A. Thomas, J. R. Slotkin, E. J. Woodard, R. Langer, Biomaterials 2011, 32, 587.
[28] M. K. Nguyen, D. K. Park, D. S. Lee, Biomacromolecules 2009, 10, 728.
[29] S. J. Rowan, S. J. Cantrill, G. R. L. Cousins, J. K. M. Sanders, J. F. Stoddart, Angew. Chem. Int. Ed., 2002, 41, 898.
[30] Voigt J et al. “Hyaluronic acid derivatives and their healing effect on burns, epithelial surgical wounds, and chronic wounds: a systematic review and meta-analysis of randomized controlled trials.” Wound Repair Regen. 2012 May-June; 20(3):317-31.
[31] K. J. Lee, D. J. Mooney, “Alginate: properties and biomedical applications”, Prog Polym Sci., 2012 January; 37(1) 106-126.
[32] (a) J. Mater. Chem. B, 2016, 4, 7050-7059; and (b) Nature Materials 8, 331-336 (2009).

1-29. (canceled)

30. (canceled)

31. A hydrogel comprising monomers of formula (I) 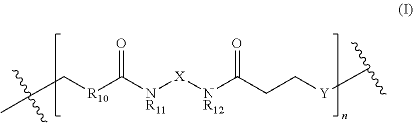
wherein
n is an integer representing the number of monomers (I) in the hydrogel polymer;
for each occurrence of the bracketed structure n:
Y independently represents:
a molecular crosslinker for connecting at least a monomer of formula (I) in the framework to at least another monomer of formula (I) in another framework through a linker having the following structure:
*—R¹-L₁-R²—*
wherein:
each occurrence of *—R¹-L₁-R²—* independently represents a responsively cleavable moiety or a non-cleavable moiety;
each occurrence of * denotes a point of attachment of the linker to a monomer of formula (I) in the hydrogel's framework;
each occurrence of L₁ independently a responsively cleavable covalent bond, a moiety containing a responsively cleavable covalent bond and/or a stable covalent bond;
R¹ and R², for each occurrence, independently represent an optionally substituted C1-20 alkylenyl moiety, an optionally substituted C1-20heteroalkylenyl moiety, an optionally substituted ethenylenyl moiety, —C≡C— or an optionally substituted phenyl moiety, wherein the C1-20 alkylenyl, C1-20 heteroalkylenyl or ethenylenyl moiety may bear one or more substituents selected from halogen or —OR where R may represent H or C1-6 allyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
wherein *—R¹-L₁-R²—* may independently comprise sugar derivatives such as mannose, hyaluronic acid derivatives, collagene, aminoacids or peptides;
or
group of formula
*—R₇(R⁸)—*
wherein
the * symbols denote the points of attachment of Y within the monomer backbone of formula (I);
R⁷ represents N,
R⁸ represents an optionally substituted C1-20 alkyl, C1-20alkenyl or C1-20alkynyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety, wherein each of the foregoing C1-20 alkyl, C1-20alkenyl, C1-20alkynyl, C1-20heteroalkyl or C1-20alkylphenyl moieties may bear one or more substituents selected from halogen, —OR, —CO₂R or —N(Rp)2; where R may represent H or C1-6alkyl and each occurrence of Rp may independently represent H or C1-6alkyl; and the phenyl moiety may bear one or more substituents independently selected from halogen, C1-6alkyl, —NO₂, —CN, isocyano, —ORp, —N(Rp)2 wherein each occurrence of Rp independently represents H, C1-6alkyl or C1-6 alkoxy; wherein R⁸ may be optionally crosslinked to another monomer of formula (I) in another hydrogel polymer chain; or
a hyaluronic acid, alginic acid, peptide, cellulose, amino acid, sugar such as glucose, lactose or mannose derivatives, or oligonucleotide moiety;
for each occurrence of the bracketed structure n, R₁₀ independently represents an optionally substituted C1-20 alkylenyl moiety, wherein the C1-20 alkylenyl moiety may bear one or more substituents selected from halogen or —OR where R may represent H or C1-6alkyl;
for each occurrence of the bracketed structure n, R₁₂, and R₁₂ independently represent H, an optionally substituted C1-20 alkyl, C1-20alkenyl or C1-20alkynyl moiety, an optionally substituted C1-20heteroalkyl moiety, or an optionally substituted phenyl moiety, wherein each of the foregoing C1-20 alkyl, C1-20alkenyl, C1-20alkynyl or C1-20heteroalkyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
for each occurrence of the bracketed structure n, X independently represents an optionally substituted C1-20 alkylenyl moiety, wherein the C1-20 alkylenyl moiety may bear one or more substituents selected from halogen or —OR where R may represent H or C1-6alkyl;
wherein at least a subset of occurrences of Y in the hydrogel polymer bears or comprises at least one organosilica particle wherein the organosilica matrix may be porous (preferably mesoporous) and contains responsively cleavable bonds within the organosilica framework.

32. The hydrogel according to claim 31, wherein at least in a subset of bracketed structures n:
L₁ represents independently a responsively cleavable covalent bond selected from: 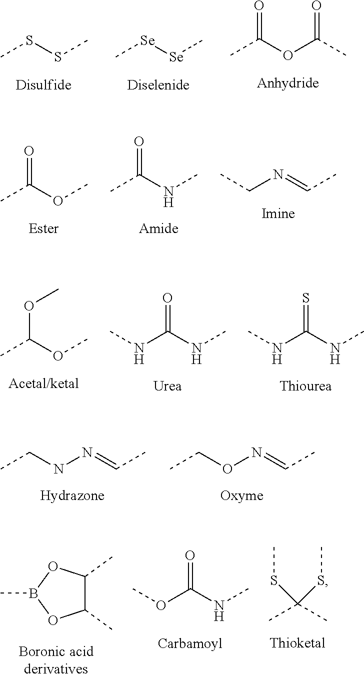
a light-induced breakable group or a photo-responsive group; or
—R¹-L₁-R²—* independently represents:
i) a pH-cleavable linker comprising to imine groups conjugated with an aromatic group such as phenyl, preferably a linker comprising a para di-imino phenyl group;
ii) a pH-cleavable linker of formula: 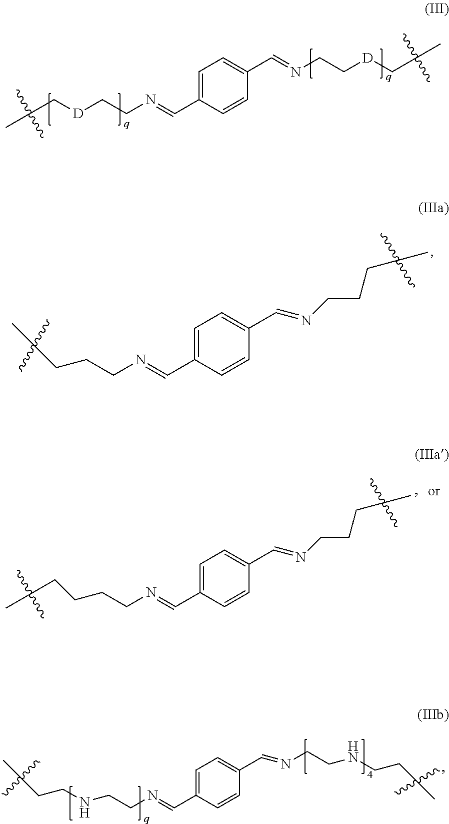
wherein each occurrence of q independently represents an integer, for example 1-6; and
D independently represents for each occurrence a C1-C3 alkylenyl moiety, or —N(Rz)- wherein Rz represents H or C1-6alkyl;
iii) a light-induced cleavable linker having formula: 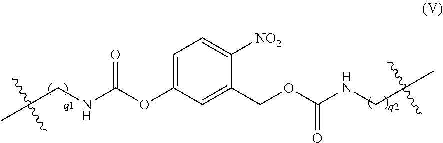
wherein q1 and q2 independently represent an integer from 1 to 6, preferably from 1 to 3.
For example, q1 and q2 may both represent an integer from 1 to 6, preferably from 1 to 3, more preferably q1=q2=3; or
iv) a responsively cleavable moiety selected from: 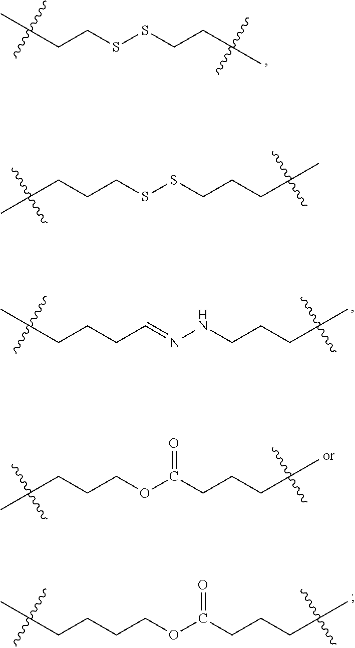 [SMILES: *CCSSCC* ; *CCCSSCCC* ; *CCC/C=N/NCCC* ; *CCCOC(=O)CCC* ; *CCCCOC(=O)CCC*]
v) a moiety comprising a sugar derivative such as mannose, a hyaluronic acid derivative, collagene, an amino acid or a peptide moiety.

33. The hydrogel according to claim 31, wherein in the linker having the structure *—R¹-L₁-R²—*, R¹ and R² are identical, and each represent —CH₂—, —(CH₂)₂—, —(CH₂)₃—, —(CH₂)₄—, or phenyl.

34. The hydrogel according to claim 31, wherein in the group of formula *—R₇(R₈)—*,
R⁷ is N and
R⁸ represents a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl.

35. The hydrogel according to claim 31, wherein in the group of formula *—R₇(R₈)—*, R⁷ may be N and R⁸ may be independently selected from the group comprising:

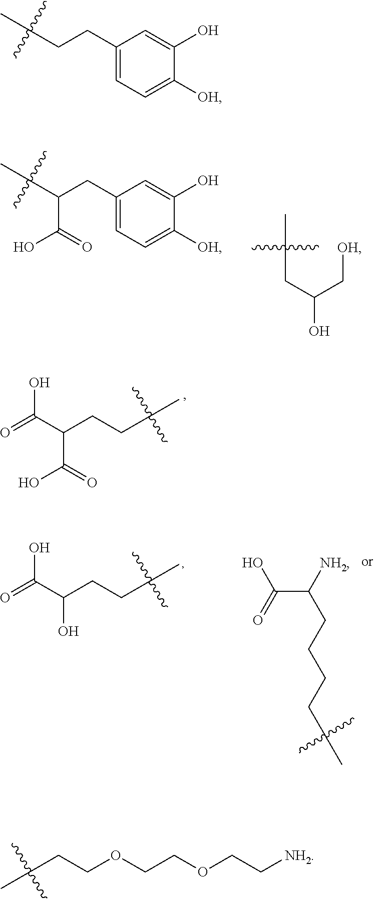

36. The hydrogel according to claim 31, wherein at least a subset of occurrences of Y in the hydrogel polymer represents *—N(R⁸)—* wherein R⁸ represents a C1-20alkyl or C1-20heteroalkyl moiety, preferably C1-6alkyl or C1-6heteroalkyl, most preferably C1-6alkyl, bearing:
(i) a disintegratable organosilica nanoparticle; or
(ii) a disintegratable organosilica nanocapsule having a core/shell structure, and a molecule of interest or bioactive macromolecule or bioactive macromolecule cluster encapsulated within said nanocapsule, wherein the bioactive macromolecule(s) or macromolecule cluster(s) within the nanocapsule is/are preferably in an active conformation;
wherein the organosilica matrix of the organosilica nanoparticle or nanocapsule contains responsively cleavable bridges #—R³-L₂-R⁴-# between Si atoms within the organosilica framework; preferably the organosilica matrix of the disintegratable organosilica nanoparticle or core/shell nanocapsule may be porous, most preferably mesoporous;
wherein:
each occurrence of # denotes a point of attachment to a Si atom in the organosilica material's framework;
L₂ represents a responsively cleavable covalent bond; and
R³ and R⁴ independently represent an optionally substituted C1-20 alkylenyl moiety, an optionally substituted C1-20 heteroalkylenyl moiety, an optionally substituted ethenylenyl moiety, —C≡C— or an optionally substituted phenyl moiety, wherein the C1-20alkylenyl, C1-20 heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —OR^{p}, —N(R^{p})₂ wherein each occurrence of R^{p}independently represents H or C1-6alkyl; and
wherein the nanoparticle or nanocapsule outer surface may comprise one or more groups of formula
#—R⁵R⁶
wherein
each occurrence of # denotes a point of attachment to a Si atom at the outer surface of the hybrid organosilica material's framework;
each occurrence of R⁵ independently represents an optionally substituted C1-20alkylenyl moiety, an optionally substituted C1-20heteroalkylenyl moiety, an optionally substituted ethenylenyl moiety, —C≡C— or an optionally substituted phenyl moiety, wherein the C1-20alkylenyl, C1-20heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO2, —CN, isocyano, —ORp, —N(Rp)2 wherein each occurrence of Rp independently represents H or C1-6alkyl; and
each occurrence of R⁶ independently represents —OR, —SR or —N(Rf)₂; preferably —N(Rf)₂; wherein each occurrence of R and Rf independently represents H or C1-6alkyl.

37. The hydrogel according to claim 31, wherein
R₁₀ represents CH— or CH—CH₂; and
R₁₁ and R₁₂ independently represent H or C1-C6 alkyl.

38. The hydrogel according to 36, wherein at least a subset of nanocapsules bound to the hydrogel polymer are further crosslinked via one or more #—R⁵R⁶ groups to another hydrogel polymer of formula I.

39. The h hydrogel according to claim 36, wherein the nanoencapsulated molecule is selected from proteins, enzymes, antibodies, peptides, DNA, RNA, PNA, gene fragments and small molecules with or without pharmaceutical activity; preferably proteins, enzymes, antibodies, peptides, DNA, RNA, PNA and gene fragments.

40. The hydrogel according to claim 36, wherein L₂ represents independently a responsively cleavable covalent bond selected from: 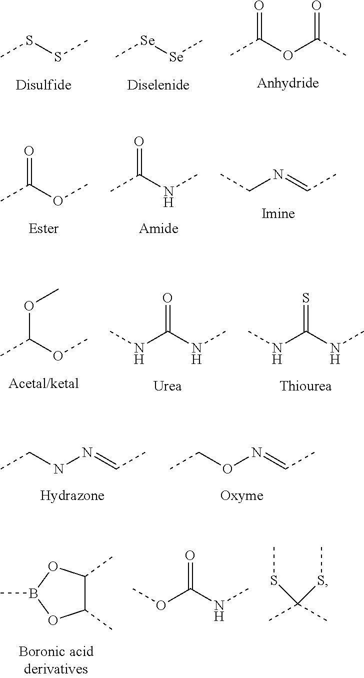
CarbamoylThioketal
a light breakable group or a photo-responsive group, or
#—R³-L₂-R⁴—# represents:
i) a pH-cleavable linker comprising to imine groups conjugated with an aromatic group such as phenyl, preferably a linker comprising a para di-imino phenyl group;
ii) a pH-cleavable linker of formula: 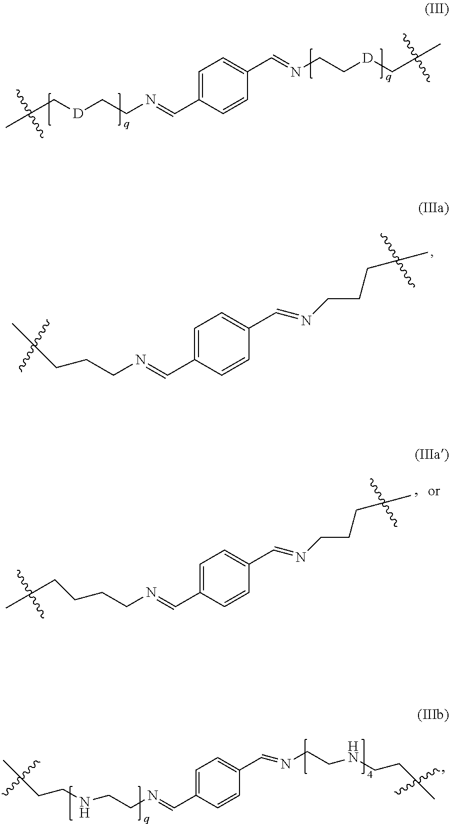
wherein each occurrence of q independently represents an integer, for example 1-6; and
D independently represents for each occurrence a C1-C3 alkylenyl moiety, or —N(Rz)- wherein Rz represents H or C1-6alkyl;
iii) a light-induced cleavable linker having formula: 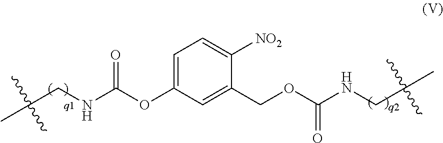
wherein q1 and q2 independently represent an integer from 1 to 6, preferably from 1 to 3.
For example, q1 and q2 may both represent an integer from 1 to 6, preferably from 1 to 3, more preferably q1=q2≤3; or
iv) a responsively cleavable moiety selected from:

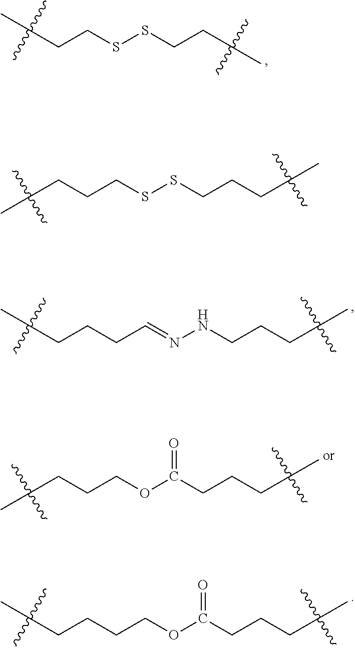 [SMILES: *CCSSCC* ; *CCCSSCCC* ; *CCC/C=N/NCCC* ; *CCCOC(=O)CCC* ; *CCCCOC(=O)CCC*]

41. The hydrogel according to claim 40, wherein the organosilica particles bound to the hydrogel polymer has a diameter between 25 nanometers and 500 nanometers.

42. A pharmaceutical or cosmetic composition comprising the hydrogel of claim 31, and a pharmaceutically or cosmetically acceptable carrier.

43. A method for preparing the hydrogel of claim 31, comprising steps of:
a) dissolving in water or alcoholic solutions:
a monomer precursor of formula (IV) 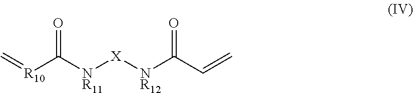
at least one molecular crosslinker precursor having the structure A-R¹-L₁-R²-A,
disintegratable organosilica nanoparticles bearing amino-containing tether groups at the outer surface; or disintegratable organosilica core/shell nanocapsules bearing amino-containing tether groups at the outer surface and encapsulating a bioactive macromolecule or bioactive macromolecule cluster, and/or another molecule of interest that may or may not have biological activity and/or pharmaceutical or cosmetic activity; wherein the bioactive macromolecule or bioactive macromolecule cluster encapsulated within the nanocapsule is preferably in active conformation; and
optionally, a selected precursor of formula B—R⁸
b) stirring the solution obtained in step a), at any appropriate temperature, thereby allowing the polymerization carried out to form the hydrogel,
c) optionally adding a suitable organic solvent, thereby precipitating the hydrogel;
wherein:
each occurrence of A independently represents a nucleophilic moiety, preferably —N(Rf)₂ wherein each occurrence of Rf may represent H or C1-6alkyl;
B independently represents a nucleophilic moiety, preferably —N(Rf)₂ wherein each occurrence of Rf may represent H or C1-6alkyl;
L₁ independently represents a responsively cleavable covalent bond, a moiety containing a responsively cleavable covalent bond and/or a stable covalent bond; and
R¹ and R² independently represent an optionally substituted C1-20 alkylenyl moiety, an optionally substituted C1-20heteroalkylenyl moiety, an optionally substituted ethenylenyl moiety, —C≡C— or an optionally substituted phenyl moiety, wherein the C1-20 alkylenyl, C1-20 heteroalkylenyl or ethenylenyl moiety may bear one or more substituents selected from halogen or —OR where R may represent H or C1-6 alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl; wherein *—R¹-L₁-R²—* may independently comprise a sugar derivative such as mannose, a hyaluronic acid derivative, collagene, an amino acid or a peptide moiety;
R₁₀ independently represents an optionally substituted C1-20 alkylenyl moiety, wherein the C1-20 alkylenyl moiety may bear one or more substituents selected from halogen or —OR where R may represent H or C1-6alkyl;
R₁₁ and R₁₂ independently represent an optionally substituted C1-20 alkyl, C1-20alkenyl or C1-20alkynyl moiety, an optionally substituted C1-20heteroalkyl moiety, or an optionally substituted phenyl moiety, wherein each of the foregoing C1-20 alkyl, C1-20alkenyl, C1-20alkynyl or C1-20heteroalkyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
X independently represents an optionally substituted C1-20 alkylenyl moiety, wherein the C1-20 alkylenyl moiety may bear one or more substituents selected from halogen or —OR where R may represent H or C1-6alkyl; and
R⁸ may independently represent:
an optionally substituted C1-20 alkyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety, wherein each of the foregoing C1-20 alkyl, C1-20heteroalkyl or C1-20alkylphenyl moieties may bear one or more substituents selected from halogen, —OR, —CO₂R or —N(Rp)2 where R may represent H or C1-6alkyl, and each occurrence of Rp may independently represent H or C1-6alkyl; and the phenyl moiety may bear one or more substituents independently selected from halogen, C1-6alkyl, —NO2, —CN, isocyano, —ORp, —N(Rp)2 wherein each occurrence of Rp independently represents H, C1-6alkyl or C1-6 alkoxy;
the residue of the corresponding amino acid H₂NR⁸;
a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl;
a C1-C6 alkyl substituted with —N(Rp)2 wherein each occurrence of Rp independently represents a C1-6alkyl;
a C2 alkyl substituted with —N(Rp)2 wherein each occurrence of Rp independently represents a C1alkyl;
a C1-20alkylphenyl moiety optionally substituted with one or more —OR wherein R may represent H or C1-6alkyl;
a group of any one of the following formulae: 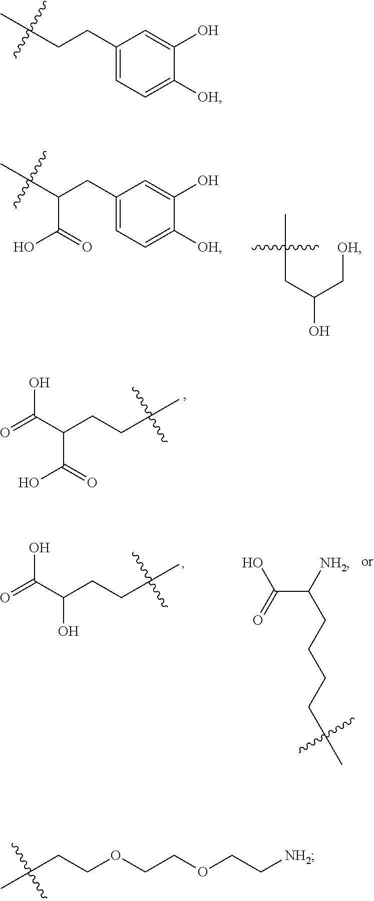
a hyaluronic acid, alginic acid, peptide, cellulose, amino acid, sugar (for example glucose, lactose or mannose derivatives) or oligonucleotide moiety; or
a C1-20alkyl or C1-20heteroalkyl moiety, preferably C1-6alkyl or C1-6heteroalkyl, most preferably C1-6alkyl, bearing an organosilica particle, preferably organosilica nanoparticles or core-shell nanocapsules, preferably the organosilica matrix may be porous, most preferably mesoporous, and may contain responsively cleavable bonds L₂ or responsively cleavable linkers #—R³-L₂-R⁴-# within the organosilica framework as defined in claim 6.

44. The method of claim 43, wherein the monomer precursor is of formula (IVa)

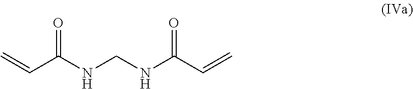

45. The method of claim 43, wherein the linker L₁ and *—R¹-L₁-R²* are as defined in claim 2.

46. The method of claim 43, wherein the molecular crosslinker precursor A-R¹-L₁-R²-A is of formula

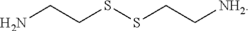

47. The method of claim 43, wherein the selected precursor of formula B—R⁸ is of formula

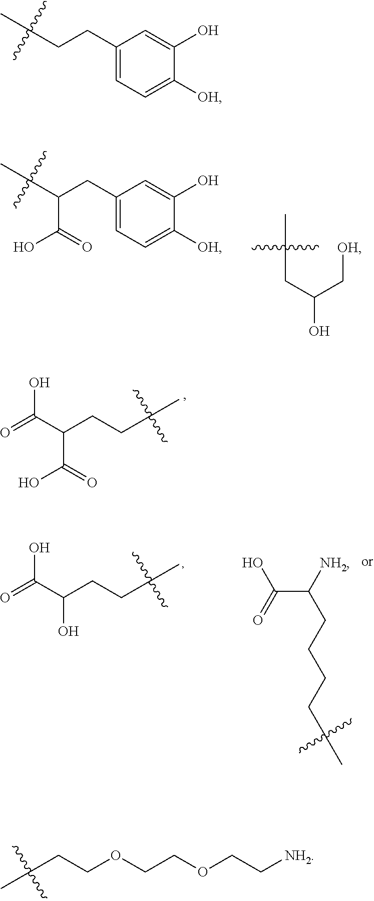

48-60. (canceled)
